(19) United States
(12) Patent Application Publication
(10) Pub. No.: US 2025/0322942 A1
(43) Pub. Date: Oct. 16, 2025

(54) COMPUTER-IMPLEMENTED, AI-BASED RECOGNITION PLATFORM

(71) Applicant: Davis Holdings of North Carolina, Inc., Cramerton, NC (US)

(72) Inventor: Charles Robert DAVIS, St. Petersburg, FL (US)

(21) Appl. No.: 19/175,315

(22) Filed: Apr. 10, 2025

Related U.S. Application Data
(60) Provisional application No. 63/632,727, filed on Apr. 11, 2024.

(51) Int. Cl. G16H 40/20; G06F 3/04817; G06F 3/0482; G06F 40/166

(52) U.S. Cl. CPC G16H 40/20; G06F 3/04817; G06F 3/0482; G06F 40/166

(57) ABSTRACT

A system for the generation of recognition notes to workers is provided. The system includes a display, one or more processors, and one or more memory devices comprising computer program code. The computer program code is configured, when executed, to cause the processor(s) to perform various tasks. These tasks include causing presentation of one or more selectable worker icons on the display, receiving an indication of a selected worker icon, causing presentation of at least two selectable statements on the display, receiving an indication of a selected statement, generating a note including the selected statement to a worker associated with the selected worker icon, and causing the note to be sent to the worker. A selectable statement of the at least two selectable statements are generated using an artificial intelligence model.

### CROSS-REFERENCE TO RELATED APPLICATION(S)

This application claims priority to U.S. Provisional Patent Application No. 63/632,727, filed Apr. 11, 2024, entitled “Computer-Implemented, AI-Based Recognition Platform,” which is hereby incorporated by reference in its entirety.

### FIELD

Embodiments relate generally to computer-implemented and artificial-intelligence based recognition platform systems and methods for using the same.

### BACKGROUND

At various workplaces such as healthcare facilities, various customers, patients, and others associated with the customers or patients often receive assistance from workers and have a high degree of appreciation for users. Despite this, these people are often unable to show their appreciation to the workers. For healthcare facilities in particular, these people and the workers frequently have busy schedules and urgent tasks that they need to focus on. Thus, even where people are appreciative of these workers, a disconnect is often present where these workers do not receive any recognition.

Additionally, employee retention and attracting new employees is difficult, particularly in the healthcare space.

### BRIEF SUMMARY

In various embodiments, recognition platform systems and methods may be provided that allow users to develop and send notes. These notes may be sent to workers such as workers at a medical facility. Users of the recognition platform system may be patients, loved ones of patients, or other visitors at a work location, and the recognition platform systems and methods may have the user identify whether he or she is a patient, a loved one, or a visitor. The user may easily identify the worker that the user is looking for. Once the worker has been identified, various statements may be presented to the user so that the user may select from statements that the user would like to include in the note. The user may in some cases be allowed to select multiple statements that the user would like to include. Various styles may also be presented that the user may select from for use in the note. Notes generated by the recognition platform system may then be sent to the relevant worker via email, text, or as a physical card that may be mailed or otherwise delivered to the relevant worker. The entire process of making a card may frequently take 60 seconds or less.

Additionally, the recognition platform system may be configured to allow administrators and operators to perform various tasks on the back end. The recognition platform system may provide analytics and generate reports based on the user data. These may allow administrators and operators to identify user preferences and to identify which statements, styles, etc. are the most popular. Analytics and reports may also provide data based on other information such as the user type for the user (patient, loved one, visitor, etc.), based on location, based on the role of the worker, etc. The recognition platform system may also allow an administrator to adjust the screens, statements, styles, etc. that are presented to the user, providing a high level of customization and freedom for the administrator.

Artificial intelligence techniques may be utilized to generate models. These models may be utilized to identify statements, styles, and other material that may be the most effective, but the models may be utilized for other purposes as well. For example, the artificial intelligence techniques may provide insights into trends or patterns in data to identify facilities (e.g., hospitals), departments, floors, areas, workers, doctors, health professionals, or other workers that are performing at a high level based on feedback from users. Artificial intelligence may also be used to analyze data, extract trends or patterns in the data, and summarize the data for administrators to review. The use of artificial intelligence techniques provides benefits to the technology, with the use of artificial intelligence providing various benefits beyond the benefits offered by basic computing systems. Artificial intelligence techniques may be used to update models over time as more data is obtained. As such, updated models may be made more accurate because they are based on a larger sample size and because they include the most recent data points, which may be entitled to greater weight than older data points in some situations. Artificial intelligence techniques may also identify patterns that humans and other computer systems may not recognize.

User interfaces are made available to users that allow users to quickly and easily navigate through screens on a display. User interfaces may be provided that are configured to be presented on small screens such as the screens of a smart phone. The user interfaces may present information to users on small screens while still making the user interfaces easy to understand and navigate. Users may easily select from several worker icons, user types, statements, styles, and other material to develop notes. Users may be allowed to filter out certain material to limit the material to be presented, making it easier for the material to be presented on a single screen. Where the material does not fit on a single screen, the user interfaces may be configured to allow the user to swipe in a certain direction to cause the material to be easily seen.

While the recognition platform systems and methods described herein are contemplated for use in hospitals and healthcare facilities, these recognition platform systems and methods may be used in other settings as well, allowing workers to receive recognition where they otherwise may not receive any. For example, recognition platform systems and methods may be used in retail settings, hospitality settings, travel settings, food service settings, dealerships, among others. By enabling employees to receive increased recognition, employers may potentially allow employees to obtain greater satisfaction from their jobs and keep more employees retained on their staff.

In an example embodiment, a system for the generation of recognition notes to workers is provided. The system comprises a display, one or more processors, and one or more memory devices comprising computer program code. The computer program code is configured, when executed, to cause the one or more processors to cause presentation of one or more selectable worker icons on the display, receive an indication of a selected worker icon, cause presentation of at least two selectable statements on the display, receive an indication of a selected statement, generate a note to a worker associated with the selected worker icon, wherein the note includes the selected statement, and cause the note to be sent to the worker. A selectable statement of the at least two selectable statements is generated using an artificial intelligence model.

In some embodiments, the computer program code may be configured, when executed, to cause the one or more processors to cause presentation of at least one selectable user type element and receive an indication of user type selection. Additionally, in some embodiments, the at least two selectable statements may depend on the indication of the user type selection that is received. In some embodiments, the system may comprise an administrator device configured to adjust the selectable worker icon(s), the at least two selectable statements, or the selectable style element(s).

In some embodiments, the system may also comprise an artificial intelligence unit configured to generate a model to identify or create at least one of a statement, a style, or a graphic for use in the note. Additionally, in some embodiments, the artificial intelligence unit may be configured to train the model using training data and deployed data, and the deployed data may be obtained after the model has been trained and deployed. Furthermore, in some embodiments, the model may be a large language model. In some embodiments, the artificial intelligence unit may be configured to receive context information and identify or create the statement, the style, or the graphic for use in the note based on the context information. The context information may include at least one of a user type, a location, the selected worker icon, or a role for a worker associated with the selected worker icon.

In another example embodiment, a system for the generation of recognition notes to workers is provided. The system includes a display, one or more processors, and one or more memory devices comprising computer program code. The computer program code is configured, when executed, to cause the one or more processors to cause presentation of one or more selectable worker icons on the display, receive an indication of a selected worker icon, cause presentation of at least two selectable statements on the display, receive an indication of a selected statement, generate a note including the selected statement to a worker associated with the selected worker icon, and cause the note to be sent to the worker.

In some embodiments, the computer program code may be configured, when executed, to cause the one or more processors to receive an indication of two or more selected statements. In some embodiments, the system may also include an administrator device configured to adjust the selectable worker icon(s), the at least two selectable statements, or the selectable style element(s).

In some embodiments, the computer program code may be configured, when executed, to cause the one or more processors to cause presentation of at least one selectable user type element and receive an indication of user type selection. Additionally, in some embodiments, the at least two selectable statements may depend on the indication of the user type selection that is received. Furthermore, in some embodiments, the computer program code may be configured, when executed, to cause the one or more processors to cause presentation of at least one selectable style element and receive an indication of a style selection, and the note may include a style associated with the style selection. In some embodiments, the artificial intelligence unit may be configured to train the model using training data and deployed data, and the deployed data may be obtained after the model has been trained and deployed.

In another example embodiment, a method for the generation of recognition notes to workers is provided. The method includes causing presentation of one or more selectable worker icons on the display, receiving an indication of a selected worker icon, causing presentation of at least two selectable statements on the display, receiving an indication of a selected statement, generating a note including the selected statement to a worker associated with the selected worker icon, and causing the note to be sent to the worker.

In some embodiments, the method may also include causing presentation of at least one selectable user type element and receiving an indication of user type selection. In some embodiments, the method may also comprise receiving context information and identifying or creating a selectable statement of the at least two selectable statements, a style, or a graphic for use in the note based on the context information. The context information may include at least one of a user type, a location, the selected worker icon, or a role for a worker associated with the selected worker icon.

### BRIEF DESCRIPTION OF THE DRAWINGS

Reference will now be made to the accompanying drawings, which are not necessarily drawn to scale, and wherein:

FIG. 1A illustrates an example screen that may be presented on a display of a user device with the screen allowing a user to specify his or her user type, in accordance with some embodiments discussed herein;

FIG. 1B is an enhanced view illustrating a first window within the screen of FIG. 1A with the first window having a graphical timeline element, in accordance with some embodiments discussed herein;

FIG. 1C is an enhanced view illustrating a second window within the screen of FIG. 1A with this second window allowing a user to specify his or her user type, in accordance with some embodiments discussed herein;

FIG. 2 illustrates an example screen that may be presented on a display of a mobile device such as smart phone with the screen allowing a user to specify his or her user type, in accordance with some embodiments discussed herein;

FIG. 3A illustrates an example screen that may be presented on a display of a user device with the screen allowing a user to identify a worker icon associated with a worker that the user wishes to provide feedback for, in accordance with some embodiments discussed herein;

FIG. 3B is an enhanced view illustrating a first window within the screen of FIG. 3A with the first window having a graphical timeline element that has advanced from a first stage to a second stage, in accordance with some embodiments discussed herein;

FIG. 3C is an enhanced view illustrating a second window within the screen of FIG. 3A with this second window allowing a user to identify a worker that the user wishes to provide feedback for, in accordance with some embodiments discussed herein;

FIG. 3D is an enhanced view illustrating an updated second window after the user has searched for an employee, with various worker icons being presented in the updated second window that the user may select, in accordance with some embodiments discussed herein;

FIG. 4 illustrates an example screen that may be presented on a display of a mobile device such as smart phone with the screen allowing a user to select a worker icon associated with a worker that the user wishes to provide feedback for, in accordance with some embodiments discussed herein;

FIG. 5A illustrates an example screen that may be presented on a display of a user device with the screen allowing the user to select statements for his or her selected worker, in accordance with some embodiments discussed herein;

FIG. 5B is an enhanced view illustrating the second window within the screen of FIG. 5A, in accordance with some embodiments discussed herein;

FIG. 6 illustrates an example screen that may be presented on a display of a mobile device such as a smart phone with the screen allowing the user to select statements for his or her selected worker, in accordance with some embodiments discussed herein;

FIG. 7A illustrates an example screen that may be presented on a display of a user device with the screen allowing the user to personalize his or her note by selecting a style for the note, in accordance with some embodiments discussed herein;

FIG. 7B is an enhanced view illustrating a second window of the screen of FIG. 7A where style selection elements may be more easily seen, in accordance with some embodiments discussed herein;

FIG. 8 illustrates an example screen that may be presented on a display of a mobile device such as a smart phone with the screen allowing the user to personalize his or her note by selecting a style for the note, in accordance with some embodiments discussed herein;

FIG. 9A illustrates an example screen that may be presented on a display of a user device allowing an administrator to view and/or adjust various settings for the recognition platform system, in accordance with some embodiments discussed herein;

FIG. 9B is an enhanced view of a first menu area of the screen of FIG. 9A, in accordance with some embodiments discussed herein;

FIG. 9C is an enhanced view of a second menu area of the screen of FIG. 9A, in accordance with some embodiments discussed herein;

FIG. 9D is an enhanced view of a logo selection area and a search option customization area of the screen of FIG. 9A, in accordance with some embodiments discussed herein;

FIG. 9E is an enhanced view an email option customization area of the screen of FIG. 9A, in accordance with some embodiments discussed herein;

FIG. 10A illustrates an example screen that may be presented on a display of a user device allowing an administrator to view and/or adjust statements that are presented to users, in accordance with some embodiments discussed herein;

FIG. 10B is an enhanced view of a statement area of the screen of FIG. 10A, in accordance with some embodiments discussed herein;

FIG. 11A illustrates an example screen that may be presented on a display of a user device allowing an administrator to view and/or adjust styles that are available for selection by users, in accordance with some embodiments discussed herein;

FIG. 11B is an enhanced view of a style area of the screen of FIG. 11A, in accordance with some embodiments discussed herein;

FIG. 12A illustrates an example report screen where statement count information is provided for each employee, in accordance with some embodiments discussed herein;

FIG. 12B illustrates an example report screen where usage information is provided for each available statement and for each persona type, in accordance with some embodiments discussed herein;

FIG. 12C illustrates an example report screen where usage information for each of the available graphics is provided, in accordance with some embodiments discussed herein;

FIG. 12D illustrates an example report screen where details on the total note count for various employees are provided, with the total note count being broken down by user type, in accordance with some embodiments discussed herein;

FIG. 12E illustrates an example report screen where details regarding unsuccessful searches are provided, in accordance with some embodiments discussed herein;

FIG. 13 is a block diagram illustrating various components of an example recognition platform system, in accordance with some embodiments discussed herein; and

FIG. 14 is a flow chart illustrating an example computer-implemented method for the creation of recognition notes to a worker, in accordance with some embodiments discussed herein.

### DETAILED DESCRIPTION

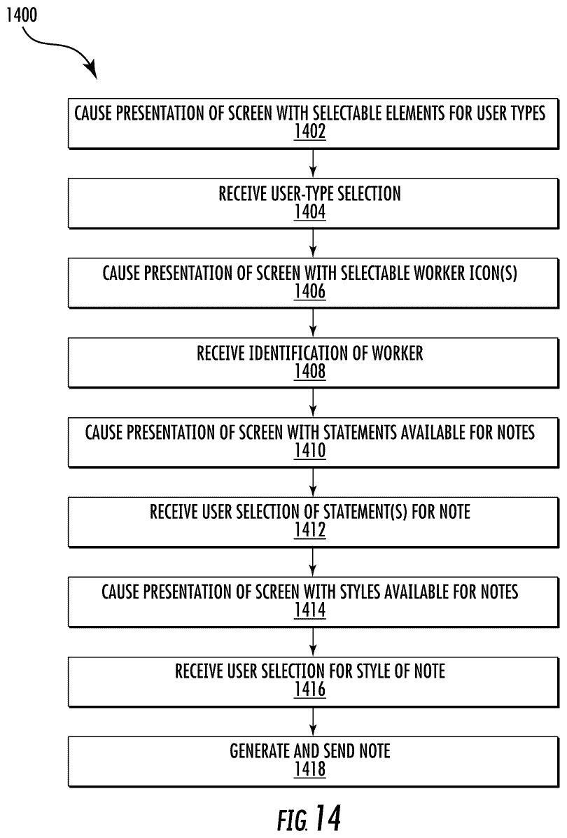

Example embodiments now will be described more fully hereinafter with reference to the accompanying drawings, in which some, but not all embodiments are shown. Other than in the flow chart of FIG. 14, like reference numerals generally refer to like elements throughout. For example, reference numbers 112, 212, 312, 412, etc. each refer to graphical timelines.

As used herein, a “worker” is a caregiver, an employee, or another non-employee working at a facility. As used herein, an “administrator” is an individual who sets up, monitors, or adjusts the recognition platform system for a particular company. An administrator may only have visibility of relevant data, reports, and information related to their company, and the administrator may have less visibility relative to an operator. As used herein, an “operator” is an individual who may have visibility of information for various companies who have set up their own respective recognition platform systems, and the operators may assist various companies and/or administrators with any issues. In some embodiments, an operator may only have visibility of information for a particular company if that company has provided express authorization.

As used herein, an “element” is a field that may be presented in a display to a user. The element may be provided in the form of a text field, an input field (e.g., a drop down input field, a push button, etc.), or some other kind of field.

As used herein, the term “artificial intelligence” is intended to mean the application of one or more software application techniques that process and analyze data to draw inferences from patterns in the data. Artificial intelligence techniques may include a variety of artificial intelligence (AI) and machine learning (ML) models or algorithms, including supervised learning techniques, unsupervised learning techniques, reinforcement learning techniques, knowledge-based learning techniques, natural-language-based learning techniques such as natural language generation and natural language processing, deep learning techniques, and the like. The artificial intelligence techniques may be trained using training data. The training data may be used to modify and fine-tune any weights associated with the machine learning models, as well as record ground truth for where correct answers may be found within the data. As such, the better the training data, the more accurate and effective artificial intelligence models may be. In some embodiments, the artificial intelligence models may be formed using a machine learning architecture. This machine learning architecture may comprise an input layer having a plurality of neurons, one or more hidden layers, with each hidden layer including a plurality of neurons, and an output layer having a one or more neurons. The neurons in the input layer may each correspond to a particular feature from a data set that is being analyzed. The neurons may provide a mathematical function that receives inputs from a previous layer, that multiplies a weight to the input based on the importance of a particular input, and that performs a summation function. Neurons may also apply activation functions in some embodiments. Central processing units (CPUs), graphical processing units (GPUs), tensor processing units, field-programmable gate arrays, application-specific integrated circuits, or other processors described herein may be utilized to implement machine learning architectures.

A large language model (“LLM”) is an artificial intelligence tool capable of generating sentences similar to how humans talk and write. Artificial intelligence techniques described herein may use LLMs in some embodiments. The LLMs may be essentially large neural networks that are trained on large amounts of data, and an example of a suitable LLM is a generative pre-trained transformer neural network model, such as GPT-3. Large language models (LLMs) may be configured to understand and generate human language by learning patterns and relationships from vast amounts of input textual data. The LLMs may utilize deep learning techniques to process and generate text. The models may be pre-trained and trained on massive data corpora (e.g., text corpora, image corpora, and the like) and may perform tasks such as text generation, image generation, sentiment analysis, and the like. The LLMs may include, by simple way of example, generative artificial intelligence (AI) or machine learning models. The generative artificial intelligence (AI) model refers to a computational system designed to create new and original data based on patterns and information learned from existing datasets. The generative AI model may employ selected machine learning techniques to generate content, such as text, images, audio, or other forms of media or data, that closely resembles the input data but is not an exact replication. The generative AI models may leverage neural networks and probabilistic methods. The LLMs may be trained or pre-trained.

The term “user interface” as used herein may refer to any software application or program, which is used to present data to an operator or end user via any selected hardware device, including a display screen, or which is used to acquire data from an operator or end user for display on the display screen. The user interface may be a series or system of interactive visual components that may be executed by suitable software. The user interface may hence include screens, windows, frames, panes, forms, reports, pages, buttons, icons, objects, menus, tab elements, and other types of graphical elements that convey or display information, execute commands, and represent actions that may be taken by the user. The objects may remain static or may change or vary when the user interacts with them. The user interface may include physical input buttons in some embodiments.

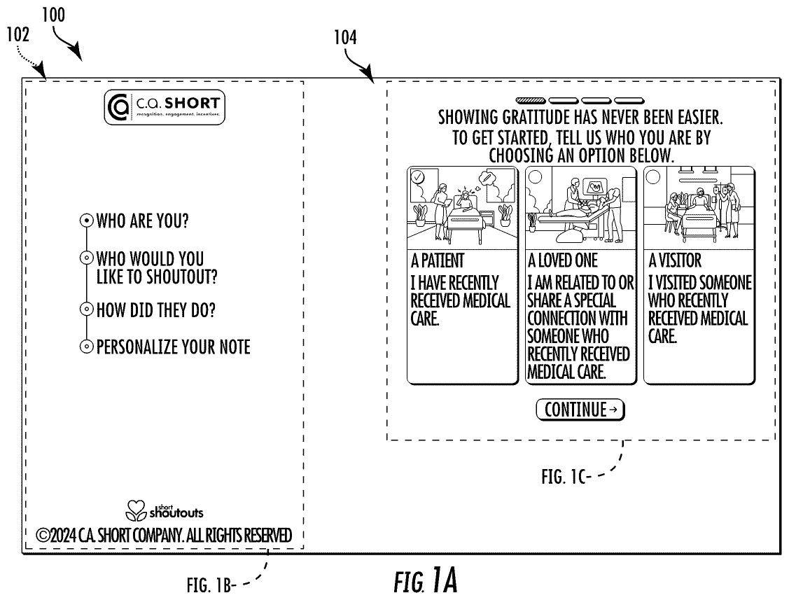

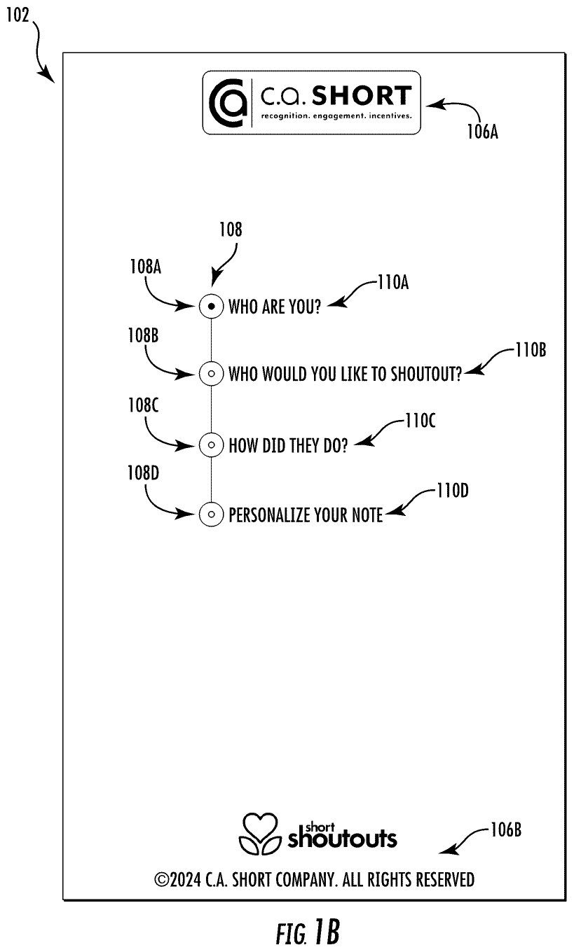

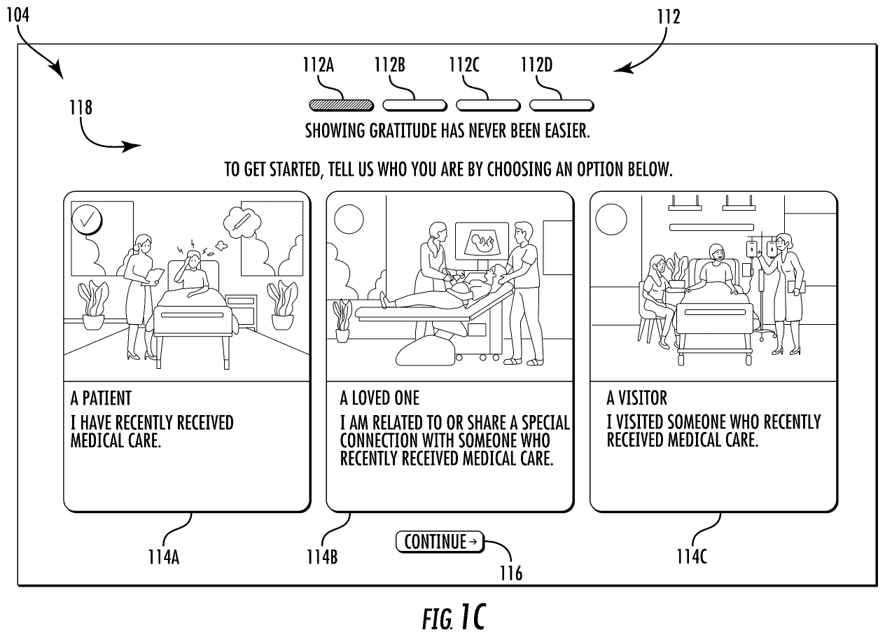

FIG. 1A illustrates an example screen 100 that may be presented on a display of a user device with the screen 100 allowing a user to specify his or her user type. The screen 100 comprises a first window 102 and a second window 104. The first window 102 is presented to the left of the second window 104 in the illustrated embodiment of FIG. 1A, but the windows 102, 104 may be positioned differently in other embodiments. For example, the first window 102 may be positioned above the second window 104, below the second window 104, or to the right of the second window 104. Alternatively, the windows 102, 104 may overlap each other, with one appearing as a pop-up window. Additionally, for other embodiments described herein, the windows may be repositioned as described in this paragraph.

Further details regarding the first window 102 may be seen in the enhanced view of FIG. 1B. The first window 102 comprises a graphical timeline 108 that provides the user with information regarding the stage of the note process that the user is on. The graphical timeline 108 includes a first timeline indicator 108A, a second timeline indicator 108B, a third timeline indicator 108C, and a fourth timeline indicator 108D. The first timeline description 110A states “Who are you?”, signifying that the first timeline indicator 108A is associated with a stage where the user type is identified for the user. The second timeline description 110B states “Who would you like to shoutout?”, signifying that the second timeline indicator 108B is associated with a stage where the worker that the user would like to provide feedback for is identified. The third timeline description 110C states “How did they do?”, signifying that the third timeline indicator 108C is associated with a stage where the user provides feedback on his or her experience with the worker. The fourth timeline description 110D states “Personalize your note”, signifying that the fourth timeline indicator 108D is associated with a stage where the user personalizes a note to the worker.

In the graphical timeline 108 presented in FIG. 1B, the first timeline indicator 108A is highlighted with a dot, and other timeline indicators 108B-108C are not highlighted with any dot. This signifies that the user is currently on the step associated with the first timeline indicator 108A (e.g., identifying who the user is or the user type for the user). However, the graphical timeline 108 and the timeline indicators 108A-108D may indicate that they are active, completed, or not-yet reached in other ways.

The first window 102 also comprises a first element 106A and a second element 106B. In FIG. 1B, the first element 106A and the second element 106B include logos, but other information may be provided in place of a logo in some embodiments. In some embodiments, the recognition platform systems may allow an administrator to change the information presented at the first element 106A and/or second element 106B to use a different logo or to use different material in place of a logo.

Further details regarding the second window 104 may be found in the enhanced view of FIG. 1C. The second window 104 allows a user to specify his or her user type. The second window 104 comprises a first selection element 114A, a second selection element 114B, and a third selection element 114C. The second window 104 also includes a description text field 118 to guide this user. The description text field 118 states “Showing gratitude has never been easier” and “to get started, tell us who you are by choosing an option below,” with the options being the selection elements 114A-114C.

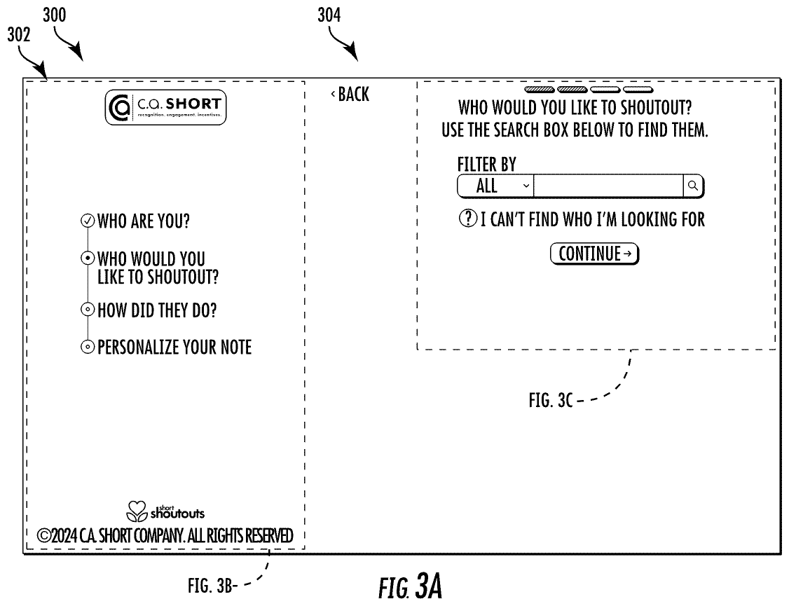

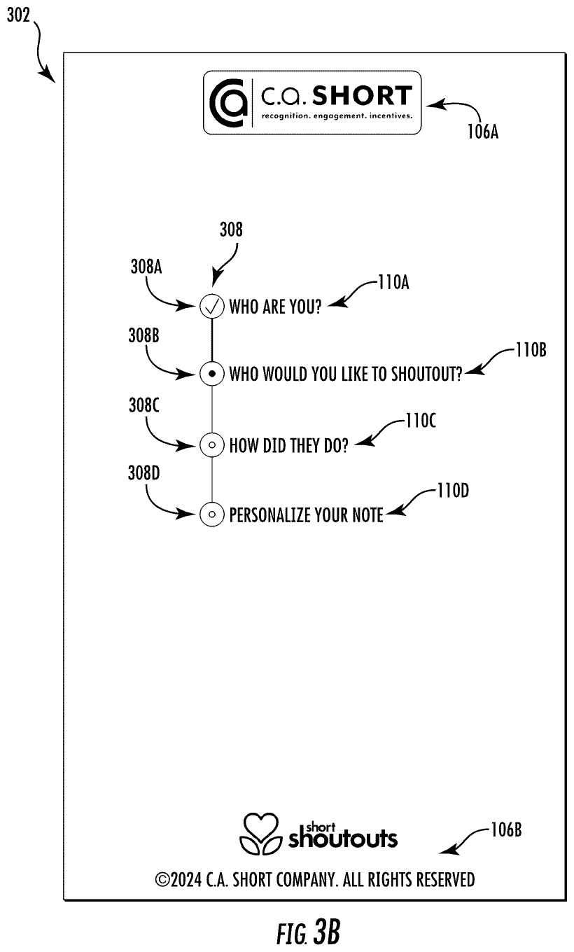

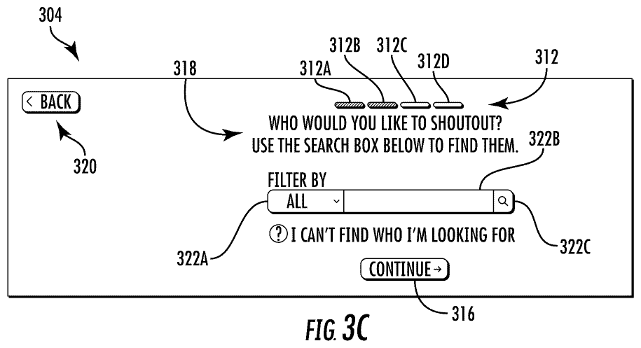

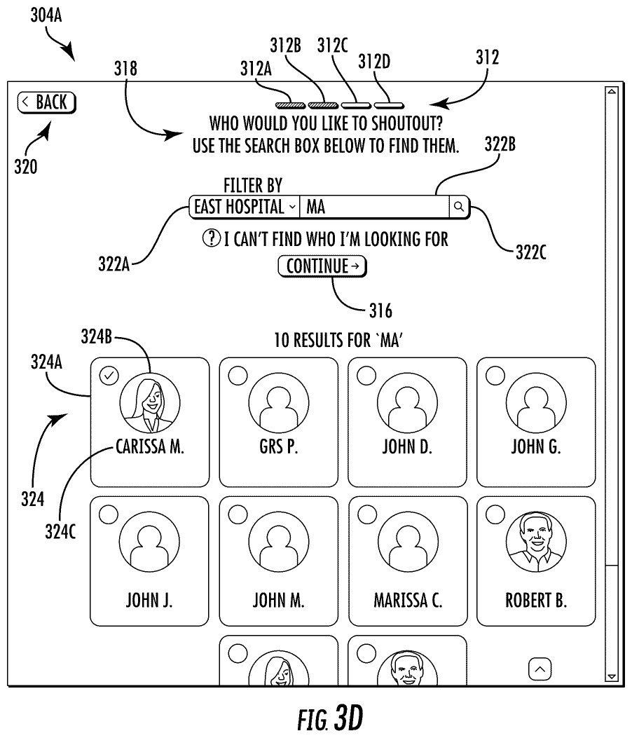

The user may select one of the selection elements 114A-114C to indicate his or her user type. In the illustrated example, the user may select the first selection element 114A to indicate that the user is a patient and that the user recently received medical care, the user may select the second selection element 114B to indicate that the user is a loved one of a patient and that the user is related to patient or shares a special connection with someone who recently received medical care, or the user may select the third selection element 114C to indicate that the user is another visitor of a patient who visited someone who recently received medical care. However, in other embodiments, a different number of selection elements may be utilized to allow for different user types. The user types may also be different in some embodiments. Once the user has selected the appropriate selection element, the user may press the continue button 116 to continue to the next screen (e.g., screen 300 of FIG. 3A).

The second window 104 also includes a graphical timeline 112. The graphical timeline includes a first graphical timeline element 112A, a second graphical timeline element 112B, a third graphical timeline element 112C, and a fourth graphical timeline element 112D. Each of the graphical timeline elements 112A-112D are lines extending colinearly. The graphical timeline elements 112A-112D may be highlighted or otherwise emphasized to indicate how far the user has gotten within the note process. For example, the first graphical timeline element 112A is highlighted while other graphical timeline elements 112B-112D remain unhighlighted, indicating that the user is on the first step. As the user progresses through additional steps, more of the timeline elements may be highlighted as well. While a graphical timeline 108 and a graphical timeline 112 are both included in the screen 100 of FIG. 1A, one or both of these graphical timelines 108, 112 may be omitted in other embodiments. Additionally, the graphical timeline 112 is merely exemplary, and it may be presented differently in other embodiments. For example, the graphical timeline elements 112A-112D may be shaped differently or emphasized differently.

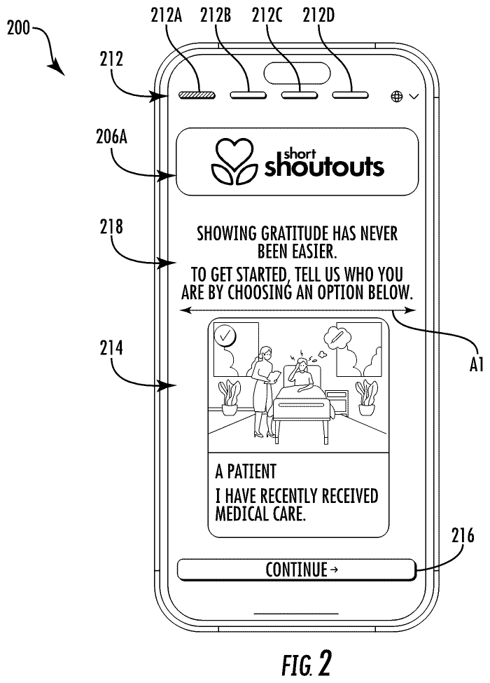

FIG. 2 illustrates an example screen 200 that may be presented on a display of a mobile device such as smart phone with the screen 200 allowing a user to specify his or her user type. The screen 200 may be similar to the second window 104 of screen 100 in FIG. 1, but screen 200 may be configured for presentation on a smaller viewing area.

The screen 200 comprises a graphical timeline 212 similar to the graphical timeline 112 of FIG. 1C. The graphical timeline 212 includes a first graphical timeline element 212A, a second graphical timeline element 212B, a third graphical timeline element 212C, and a fourth graphical timeline element 212D. Each of the graphical timeline elements 212A-212D are lines extending colinearly. The graphical timeline elements 212A-212D may be highlighted or otherwise emphasized to indicate how far the user has gotten within the note process. For example, the first graphical timeline element 212A is highlighted while other graphical timeline elements 212B-212D remain unhighlighted, indicating that the user is on the first step. As the user progresses through additional steps, more of the timeline elements may be highlighted as well. Additionally, the graphical timeline 212 is merely exemplary, and it may be presented differently in other embodiments. For example, the graphical timeline elements 212A-212D may be shaped differently or emphasized differently.

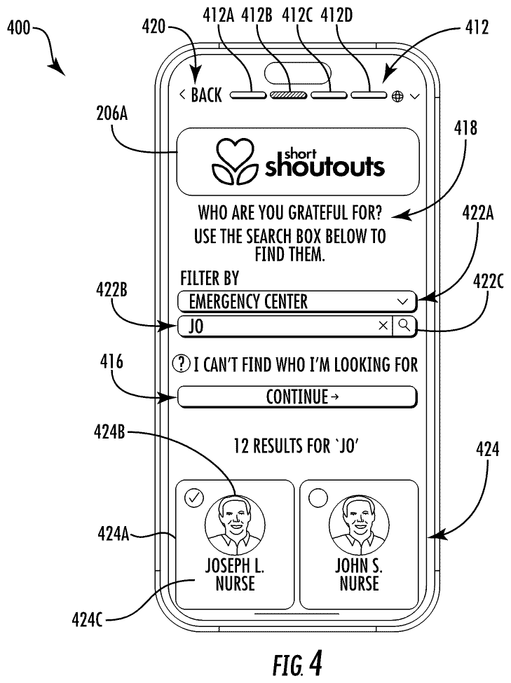

The screen 200 allows a user to specify his or her user type. In FIG. 2, the screen 200 may present selection elements 214. While only one selection element 214 is illustrated in FIG. 2, the user may optionally swipe the screen to the left and/or to the right (as indicated by the line A1) to cause other selection elements to be presented. The selection elements 214 available for presentation on the screen 200 may be similar to the selection elements 114A-114C described above, but other selection elements may be used as well. The screen 200 also includes a description text field 218 to guide the user. The description text field 218 states “to get started, tell us who you are by choosing an option below,” with the options being the selection elements 214. The user may select one of the selection elements 214 to indicate his or her user type. Once the user has selected the appropriate selection element, the user may press the continue button 216 to continue to the next screen (e.g., screen 400 of FIG. 4). An element 206A is also illustrated in FIG. 2. This element 206A is a logo in FIG. 2, but other information may be provided in place of a logo in some embodiments. In some embodiments, the recognition platform systems may allow an administrator to change the information presented at the element 206A to use a different logo or to use different material in place of a logo.

FIG. 3A illustrates an example screen 300 that may be presented on a display of a user device with the screen 300 allowing a user to identify a worker that the user wishes to provide feedback for. The screen 300 comprises a first window 302 and a second window 304.

Further details regarding the first window 302 may be seen in the enhanced view of FIG. 3B. The first window 302 is similar in many respects to the first window 102 of FIG. 1B, with similar graphical timelines 108, 308 included in both. However, in FIG. 3B, the first timeline indicator 308A includes a checkmark to indicate that the first stage of the note process has been completed, the second timeline indicator 308B includes a highlighted dot to indicate that the second stage is the stage that the user is actively working on, and the third and fourth timeline indicators 308C, 308D do not provide a checkmark or a highlighted dot, indicating that the third and fourth stages have not yet been reached.

Further details regarding the second window 304 may be seen in the enhanced view of FIG. 3C. The second window 304 allows a user to identify a worker that the user wishes to provide feedback for.

The second window 304 includes a graphical timeline 312. The graphical timeline 312 includes a first graphical timeline element 312A, a second graphical timeline element 312B, a third graphical timeline element 312C, and a fourth graphical timeline element 312D. The graphical timeline 312 is generally similar to the graphical timeline 112 of FIG. 1C. However, in the graphical timeline 312 of FIG. 3C, both the first and second graphical timeline elements 312A, 312B are highlighted to indicate that the user is at the second stage of the note process. The third and fourth graphical timeline elements 312C, 312D remain unhighlighted.

The second window 304 also includes a description text field 318 to guide this user. The description text field 318 states “Who would you like to shoutout?” and Use the search box below to find them.” The user may then use the fields 322A, 322B to identify the worker. The filter selection field 322A may allow the user to filter certain workers out to focus on a particular subgroup of workers (e.g., dayshift, nightshift, working in a particular part of a healthcare facility, etc.). The filter selection field 322A is provided in the form of a drop-down menu where a pre-set list of options is provided to the user for selection. The input field 322B also allows the user to input information to search for the appropriate worker. A user may freely type text within the input field 322B. However, in other embodiments, the type of fields for fields 322A, 322B may be different. The user may select the search button 322C after inputting material at the field 322A or input field 322B. However, in some embodiments, the user may select the search button 322C without inputting any material at fields 322A, 322B.

Various worker icons may be visible to the user after the user selects the search button 322C. The updated second window 304A of FIG. 3D illustrates an example of this. The updated second window 304A illustrates a plurality of worker icons 324, including a first worker icon 324A and other worker icons. The first worker icon 324A includes an avatar 324B and a worker identifier 324C. The avatar 324B may be an image of the user, a generic placeholder image where no other image has been provided or selected, or some other image or symbol. The worker identifier 324C may be the name of the worker, a role of the worker, a location of the worker, a facility of the worker, a department of the worker, etc. Where the name of the worker is used as the worker identifier 324C as illustrated in FIG. 3D, the name of the user may be presented in different ways. In some embodiments, only the first name may be presented. In others, the first name and last name may be presented or the first name and the last initial may be included. In some embodiments, worker icons may automatically appear on the second window 304 before the user ever selects the search button 322C. In some embodiments, the user may only select one worker icon. However, in other embodiments, the user may select multiple worker icons and may create multiple notes to these workers at the same time—where this is the case, the notes may be identical or the notes may alternatively be customized for the different workers. In some embodiments, the worker icons may be sorted based on various factors, including the number of times a particular worker associated with a worker icon has received a note, whether the worker is typically interacting with only other staff members or with patients, visitors, or others, etc. In some embodiments, artificial intelligence techniques may be used to sort the worker icons in an optimal order based on various input factors.

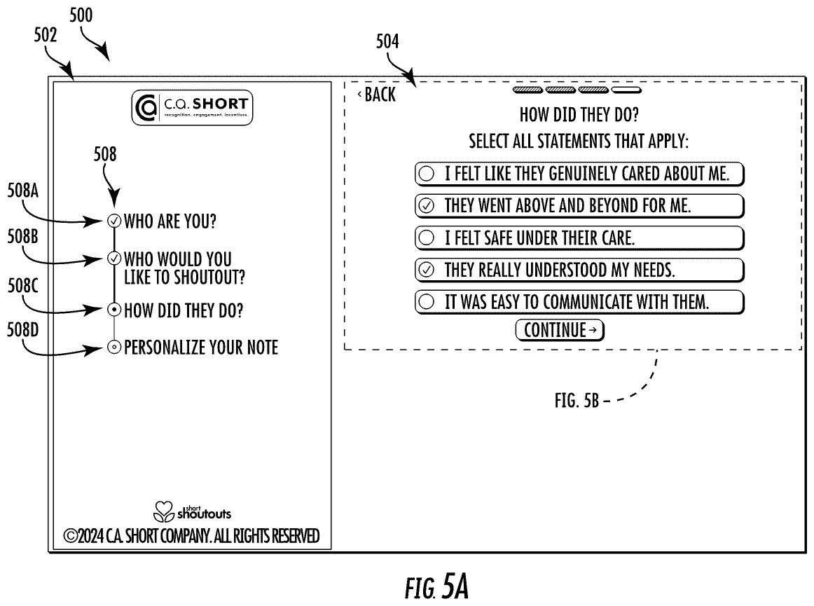

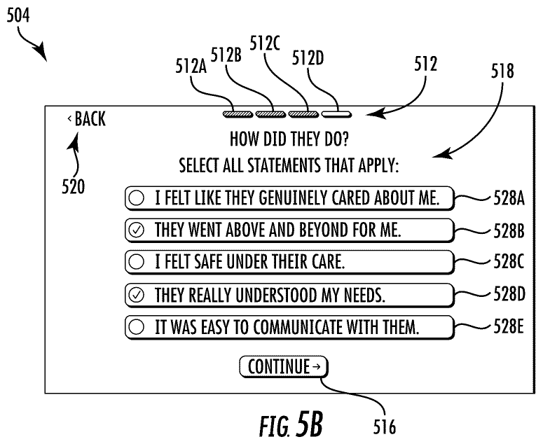

In both FIGS. 3C and 3D, the windows 304, 304A include a back button 320 and a continue button 316. Selection of the back button 320 may cause a previously presented screen (e.g., screen 100 of FIG. 1A) to appear, and selection of the continue button 316 may cause the system to advance to the next screen (e.g., screen 500 of FIG. 5A).

FIG. 4 illustrates an example screen 400 that may be presented on a display of a mobile device such as smart phone, with the screen 400 allowing a user to identify a worker icon associated with a worker that the user wishes to provide feedback for. The screen 400 may be similar to the second window 304 of screen 300 in FIGS. 3A and 3C, but screen 400 may be configured for presentation on a smaller viewing area.

The screen 400 is similar in many respects to the screen 200 of FIG. 2. Like screen 200, screen 400 includes a graphical timeline 412. The graphical timeline 412 includes a first graphical timeline element 412A, a second graphical timeline element 412B, a third graphical timeline element 412C, and a fourth graphical timeline element 412D. The graphical timeline 412 is generally similar to the graphical timeline 212 of FIG. 2. However, in the graphical timeline 412 of FIG. 4, both the second graphical timeline element 412B is highlighted to indicate that the user is at the second stage of the note process. The, first, third, and fourth graphical timeline elements 412A, 412C, 412D remain unhighlighted.

The screen 400 also includes a description text field 418 to guide this user. The description text field 418 states “Who are you grateful for? Use the search box below to find them.” The user may then use the fields 422A, 422B to identify the worker. The filter selection field 422A may allow the user to filter certain workers out to focus on a particular subgroup of workers (e.g., dayshift, nightshift, working in a particular part of a healthcare facility, etc.). The filter selection field 422A is provided in the form of a drop-down menu where a pre-set list of options is provided to the user for selection. The input field 422B also allows the user to input information to search for the appropriate worker. A user may freely type text within the input field 422B. However, in other embodiments, the type of fields for fields 422A, 422B may be different. The user may select the search button 422C after inputting material at the field 422A or field 422B. However, in some embodiments, the user may select the search button 422C without inputting any material at fields 422A, 422B.

Once the user selects the search button 422C, a list of worker icons 424 may be made visible to the user or the list of worker icons 424 may be updated. The worker icons 424 include a first worker icon 424A and other worker icons. The system may be configured to allow the user to swipe in a certain direction (e.g., up, down, left, right, etc.) to cause different worker icons 424 to be made visible. The first worker icon 424A includes an avatar 424B and a worker identifier 424C. The avatar 424B may be an image of the user, a generic placeholder image where no other image has been provided or selected, or some other image or symbol. The worker identifier 424C may include the name of the worker, a role of the worker, a location of the worker, a facility of the worker, a department of the worker, etc. Where the name of the worker is used as the worker identifier 424C as illustrated in FIG. 4, the name of the user may be presented in different ways. In some embodiments, only the first name may be presented. In others, the first name and last name may be presented or the first name and the last initial may be included.

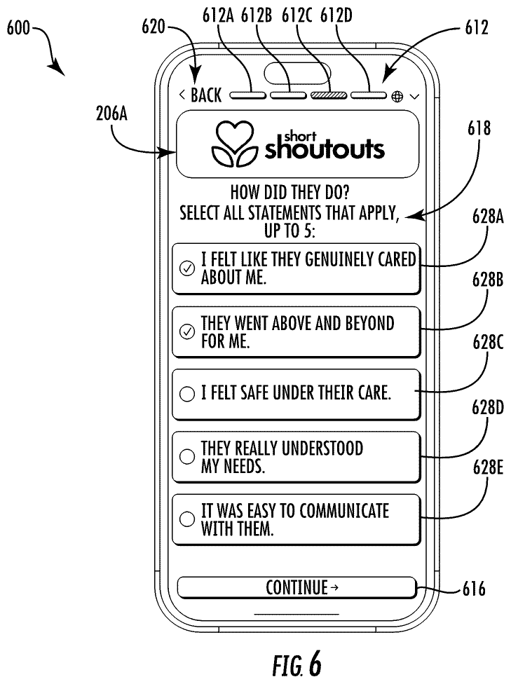

In FIG. 4, the screen 400 includes a back button 420 and a continue button 416. The user may press the continue button 416 to proceed to the next screen (e.g., screen 600 of FIG. 6). The user may also select the back button 420 to return to an earlier screen (e.g., screen 200 of FIG. 2).

FIG. 5A illustrates an example screen 500 that may be presented on a display of a user device with the screen 500 allowing the user to select statements for his or her selected worker. The selected statements or other similar statements may eventually be used in the notes that are formed.

The screen 500 includes a first window 502 and a second window 504. The first window 502 is similar to the first windows 102, 302 of FIGS. 1B and 3B, with the first window 502 similarly including a graphical timeline 508 comprising a first timeline indicator 508A, a second timeline indicator 508B, a third timeline indicator 508C, and a fourth timeline indicator 508D. However, the graphical timeline 508 indicates that the user is at the third stage. The first and second timeline indicators 508A, 508B both include a checkmark to indicate that the first and second stages of the note process have been completed, the third timeline indicator 508C includes a highlighted dot to indicate that the third stage is the stage that the user is actively working on, and the fourth timeline indicator 508D does not provide a checkmark or a highlighted dot, indicating that the fourth stage has not yet been reached.

Further details regarding the second window 504 may be seen in the enhanced view of FIG. 5B. The second window 504 allows a user to identify a worker that the user wishes to provide feedback for. The second window 504 comprises a description text field 518 stating “How did they do? Select all statements that apply:”. The second window 504 also comprises several statement selection elements for the user to select from, including a first statement selection element 528A, a second statement selection element 528B, a third statement selection element 528C, a fourth statement selection element 528D, and a fifth statement selection element 528E. The statement selection elements 528A-528E provide various statements that the user may utilize for a note to a worker. While five statement selection elements are included in the second window 504 of FIG. 5B, a different number of statement selection elements may be included in the second window 504 in other embodiments.

In the illustrated embodiment of FIG. 5B, the first statement selection element 528A states “I felt like they genuinely cared about me.” The second statement selection element 528B states “They went above and beyond for me.” The third statement selection element 528C states “I felt safe under their care.” The fourth statement selection element 528D states “They really understood my needs.” The fifth statement selection element 528E states “It was easy to communicate with them.” However, the statements for each of the statement selection elements 528A-528E may be different in other embodiments. In the illustrated embodiment, the user has selected the second and fourth statement selection elements 528B, 528D, and the first, third, and fifth statement selection elements 528A, 528C, 528E remain unselected.

The statement selection elements 528A-528E may be generated through the use of artificial intelligence (e.g., generative artificial intelligence) using an artificial intelligence unit in some embodiments. For example, the statement selection elements 528A-528E may be generated through the use of LLMs. By using artificial intelligence, statement selection elements may be automatically used that are most appealing to users, that are most appropriate, or that are the best under some other criteria. Data may be obtained from previous users to identify statements that are most appealing to users, that are most appropriate, etc., with this data being used to train an artificial intelligence model. The data may be limited to previous users of a recognition platform system for a particular company, but the data may be obtained from previous users of recognition platform systems of several different companies to increase the size of the available data set, thereby allowing potentially more accurate models to be created. Where data is obtained from recognition platform systems of several different companies, this data may not contain any personal identifying information or private health information.

The use of artificial intelligence tools may reduce the amount of time that the administrator must take to make the system available to the public. Alternatively, other computer based approaches may be used to automatically generate statement selection elements. Artificial intelligence or other computer based approaches may utilize the available context to identify statements for use in statement selection elements that are of particular relevance to the context. For example, the artificial intelligence or other computer based approaches may rely on context such as the responses submitted by the user, the user type (e.g., patient, caregiver, etc.), the worker that was identified, the location where the user visited or where the worker is located, a focus area for the worker, demographic information for the worker, etc. For example, where the user is a loved one and where the identified worker works in a children's hospital, the statements used in statement selection elements may be different than another context where the user is a patient and where the identified worker works in a dentist's office. While the context may consider demographic information and other information for the user, the patient, and/or the worker in some embodiments, this information may not be considered in other embodiments to avoid the use and/or disclosure of personal identifying information or private health information.

In some embodiments, artificial intelligence or other computer based approaches may merely generate suggested statements for use in statement selection elements and an administrator may select statements from the suggested statements that the administrator would like to use. While the use of artificial intelligence and computer based approaches are described herein to help identify appropriate statements to include in notes, artificial intelligence and computer based approaches may also be used to identify styles, graphics, and other properties that may be ideal for use in notes. Artificial intelligence and computer based approaches may also be used for other purposes.

In some embodiments, the user may select multiple statement selection elements. For example, the user may select more than one statement selection element but less than all of the statement selection elements in some embodiments. Alternatively, the user may select all of the statement selection elements in some embodiments or the user may select only one of the statement selection elements.

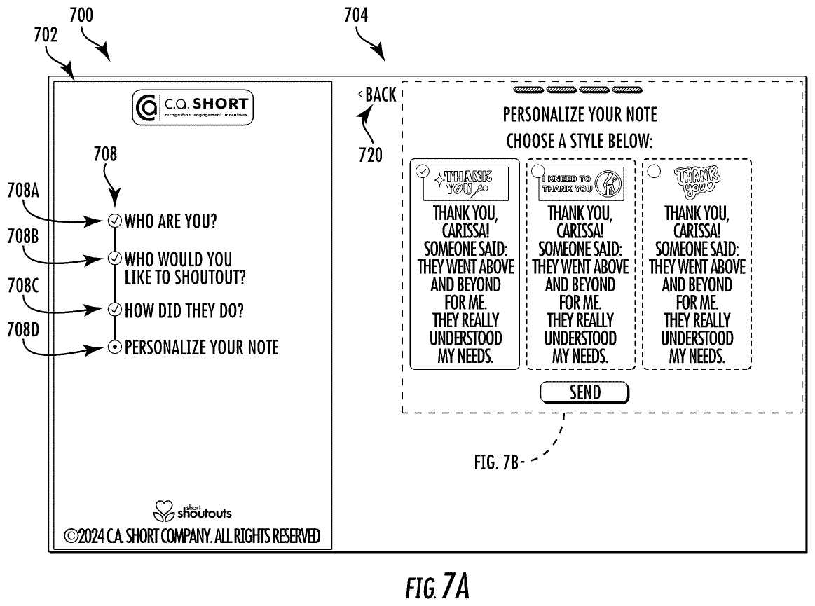

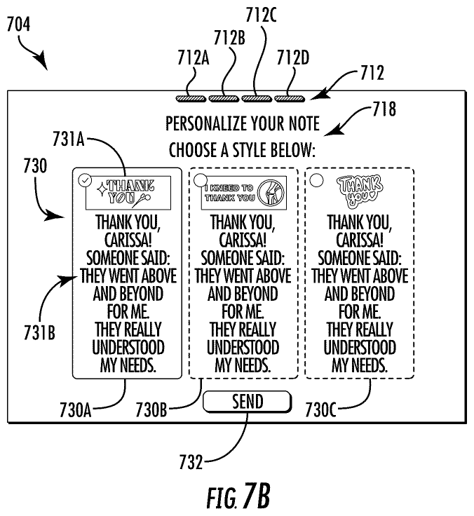

The second window 504 also includes a graphical timeline 512 having graphical timeline elements 512A-512D to indicate the stage of the process (e.g., the third stage in the illustrated embodiment). Once the user has selected the statement selection element(s), the user may press the continue button 516 to proceed to the next screen (e.g., screen 700 of FIG. 7A). The user may also select the back button 520 to return to an earlier screen (e.g., screen 300 of FIG. 3A).

FIG. 6 illustrates an example screen 600 that may be presented on a display of a mobile device such as smart phone with the screen 600 allowing a user to select statements for his or her selected worker. The screen 600 may be similar to the second window 504 of screen 500 in FIGS. 5A and 5B, but screen 600 may be configured for presentation on a smaller viewing area. Like the second window 504 of screen 500, screen 600 includes a description text field 618. The description text field 618 states “How did they do?” and “Select all statements that apply, up to 5:”. Similar to the second window 504 of screen 500, screen 600 also includes a first statement selection element 628A, a second statement selection element 628B, a third statement selection element 628C, a fourth statement selection element 628D, and a fifth statement selection element 628E. These statement selection elements 628A-628E include statements that are identical to the statements of statement selection elements 528A-528E. In some embodiments, the statement selection elements 628A-628E may have a size and shape that allows these elements to be positioned on a smaller screen for the mobile device.

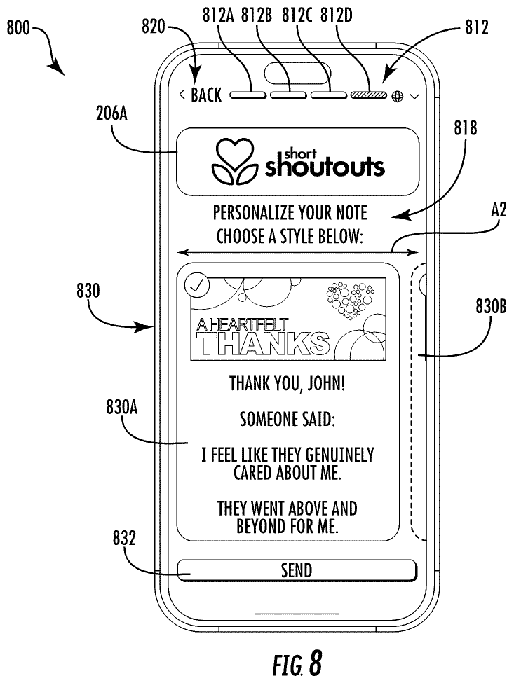

The screen 600 includes a graphical timeline 612 similar to previously described graphical timelines. The graphical timeline 612 has graphical timeline elements 612A-612D that indicate the stage of the process (e.g., the third stage in the illustrated embodiment). The user may press the continue button 616 to proceed to the next screen (e.g., screen 800 of FIG. 8). The user may also select the back button 620 to return to an earlier screen (e.g., screen 400 of FIG. 4).

FIG. 7A illustrates an example screen 700 that may be presented on a display of a user device with the screen 700 allowing the user to personalize his or her note by selecting a style for the note. The screen 700 includes a first window 702 and a second window 704. The second window 704 includes a back button 720, and a user may select the back button 720 to return to an earlier screen (e.g., screen 500 of FIG. 5A). The first window 702 is similar to the first windows 102, 302, 502 of FIGS. 1B, 3B, and 5A, with the first window 702 similarly including a graphical timeline 708 comprising a first timeline indicator 708A, a second timeline indicator 708B, a third timeline indicator 708C, and a fourth timeline indicator 708D. However, the graphical timeline 708 indicates that the user is at the fourth stage. The first, second, and third timeline indicators 708A, 708B, 708C each include a checkmark to indicate that the first three stages of the note process have been completed, the fourth timeline indicator 708D includes a highlighted dot to indicate that the fourth stage is the stage that the user is actively working on.

Further details regarding the second window 704 may be seen in the enhanced view of FIG. 7B. The second window 704 allows a user to personalize his or her note by selecting a style for the note.

The second window 704 comprises a description text field 718 stating “Personalize your note” and “Choose a style below:”. The second window 704 also comprises style selection elements 730 for the user to select from, including a first style selection element 730A, a second style selection element 730B, and a third style selection element 730C. The style selection elements 730 provide styles that the user may utilize for a note to a worker. For example, the first style selection element 730A includes a graphic 731A and a text field 731B. The graphic 731A for the first style selection element 730A is different from graphics used in other style selection elements. The graphics and style selection elements 730 are merely exemplary, and other graphics and style selection elements 730 may be used. The style selection elements 730 may be generated through the use of artificial intelligence (e.g., generative artificial intelligence) using an artificial intelligence unit in some embodiments. For example, the style selection elements 730 may be generated through the use of LLMs or other generative artificial intelligence techniques. By using artificial intelligence, style selection elements may be automatically used that are most appealing to users, that are most appropriate, or that are the best under some other criteria. Data may be obtained from previous users to identify style selection elements that are most appealing to users, that are most appropriate, etc., with this data being used to train an artificial intelligence model. The data may be limited to previous users of a recognition platform system for a particular company, but the data may be obtained from previous users of recognition platform systems of several different companies to increase the size of the available data set, thereby allowing potentially more accurate models to be created.

Additionally, the text field 731B includes statements similar to those previously selected by the user at an earlier screen. In some embodiments, the style selection elements may be presented with different fonts, font sizes, text colors, spacing, background colors, background images, etc. In other embodiments, additional fields may be provided in the second window 704 to enable the user to adjust the style for notes (e.g., to adjust the font, font size, text color, spacing, background color, background image, etc.). Once the user has finished selecting and/or adjusting the style for their note, the user may press the send button 732 to cause his or her note to be sent. Once sent, the note may be provided to the identified worker. Additionally, the note may be sent to a manager or supervisor of the employer or to others.

The second window 704 comprises a graphical timeline 712 that is similar to previously described graphical timelines. The graphical timeline 712 has graphical timeline elements 712A-712D that indicate the stage of the process (e.g., the fourth stage in the illustrated embodiment).

FIG. 8 illustrates an example screen 800 that may be presented on a display of a mobile device such as smart phone with the screen 800 allowing a user to personalize his or her note by selecting a style for the note. The screen 800 may be similar to the second window 704 of screen 700 in FIGS. 7A and 7B, but screen 800 may be configured for presentation on a smaller viewing area.

The screen 800 comprises a description text field 818 stating “Personalize your note” and “Choose a style below:” The screen 800 also comprises style selection elements 830 for the user to select from, including a first style selection element 830A and a second style selection element 830B. While only two style selection elements 830 are visible in FIG. 8, additional style selection elements may be made visible if the user swipes in a certain direction. For example, if the user swipes left or right as indicated by line A2, then different style selection elements may be made visible. The style selection elements 830 provide styles that the user may utilize for a note to a worker. The style selection elements 830 may be similar to the style selection elements 730 of FIG. 7B.

The screen 800 includes a graphical timeline 812 similar to previously described graphical timelines. The graphical timeline 812 has graphical timeline elements 812A-812D that indicate the stage of the process (e.g., the fourth stage in the illustrated embodiment).

In FIG. 8, the screen 800 includes a back button 820 and a send button 832. The user may press the send button 832 to cause his or her note to be sent. Once sent, the note may be provided to the identified worker. Additionally, the note may be sent to a manager or supervisor of the employer or to others. The user may also select the back button 820 to return to an earlier screen (e.g., screen 600 of FIG. 6).

In some embodiments, screens described herein may be provided as part of a software application or program, and the user may access the software application or the program in various ways. For example, the user may scan a QR code, the user may navigate to a URL, the user may receive a link from the company or the healthcare facility that directs the user to the software application or program, etc.

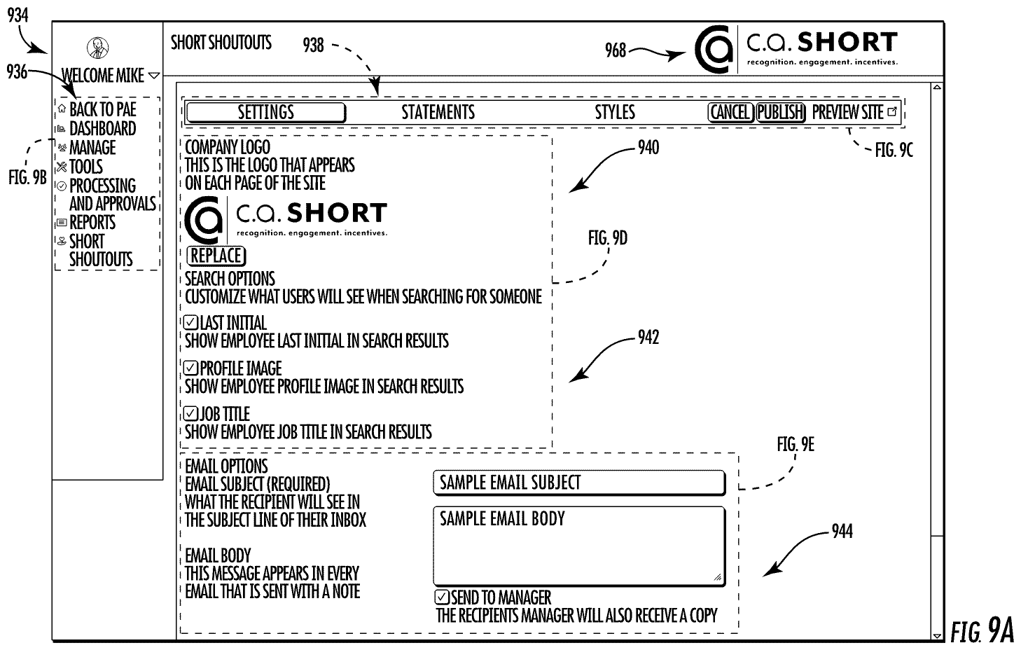

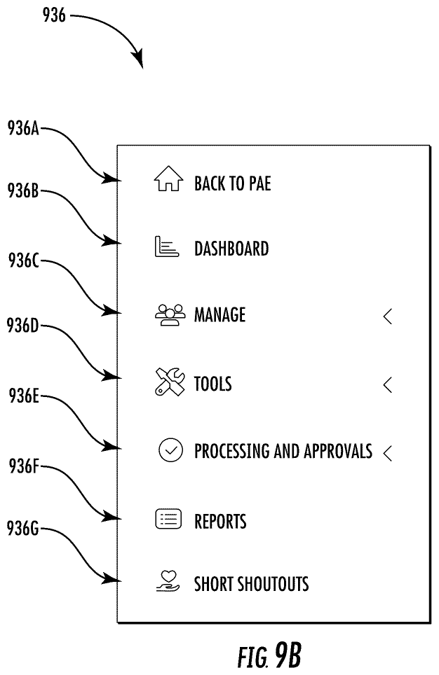

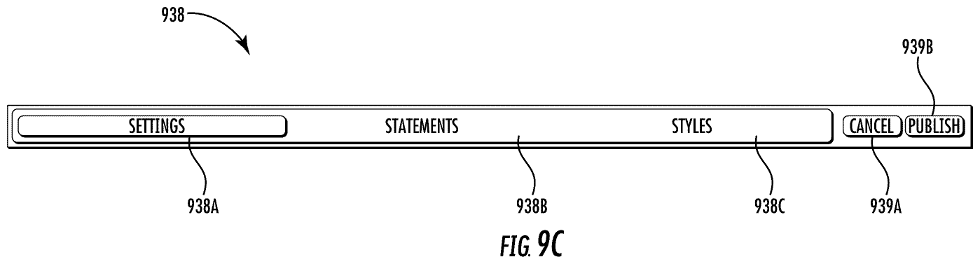

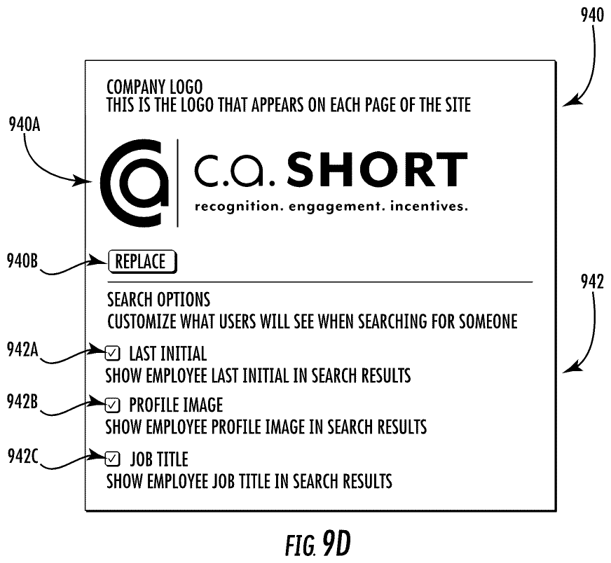

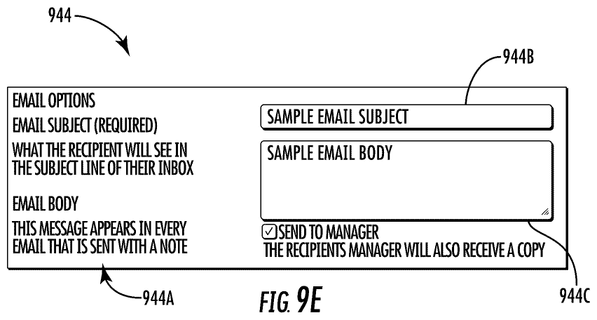

In various embodiments, the recognition platform system may also allow an administrator or an operator to monitor the recognition platform system and to make adjustments in the recognition platform system. FIG. 9A illustrates an example screen 934 that may be presented on a display of a user device allowing an administrator or operator to view and/or adjust various settings for the recognition platform system. The screen 934 includes a first menu area 936, a second menu area 938, a logo selection area 940, a search option customization area 942, and an email option customization area 944. Each of these areas are described in greater detail herein.

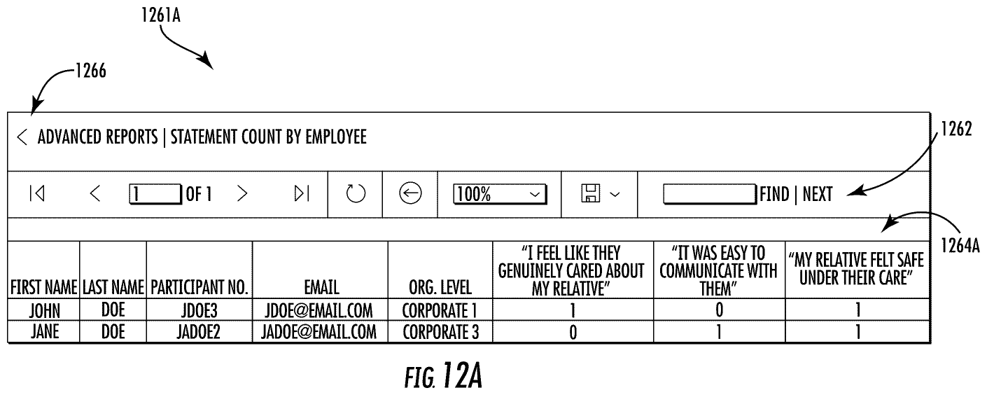

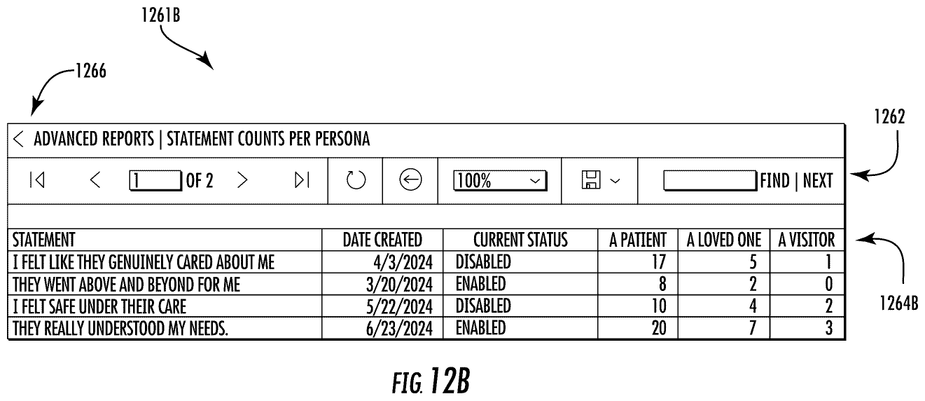

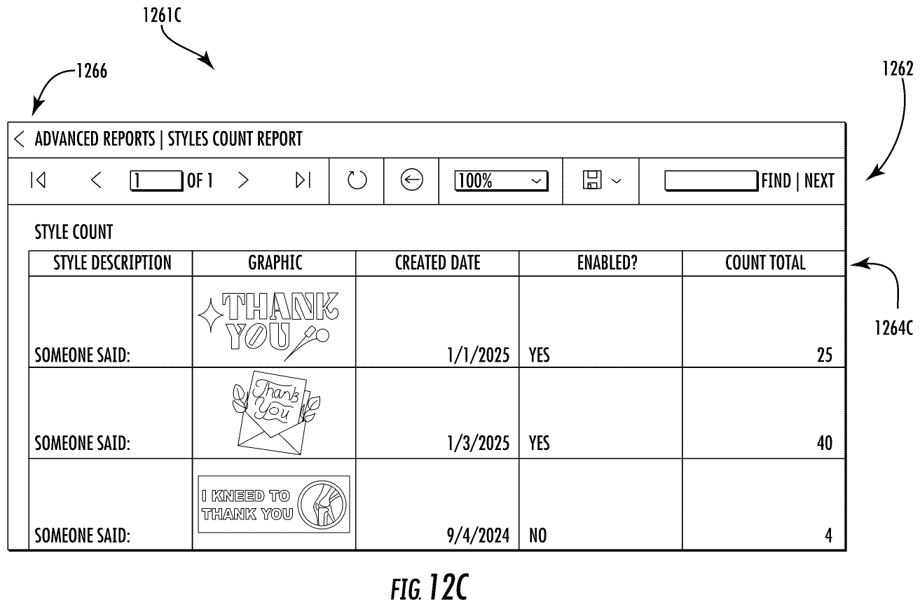

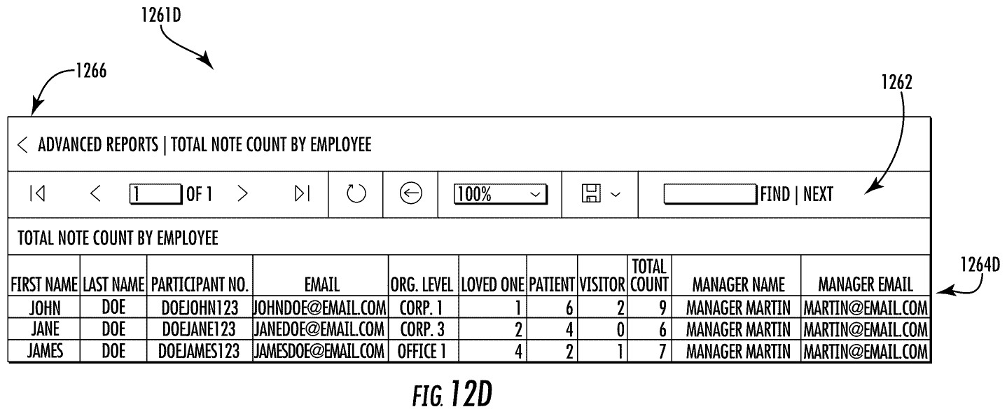

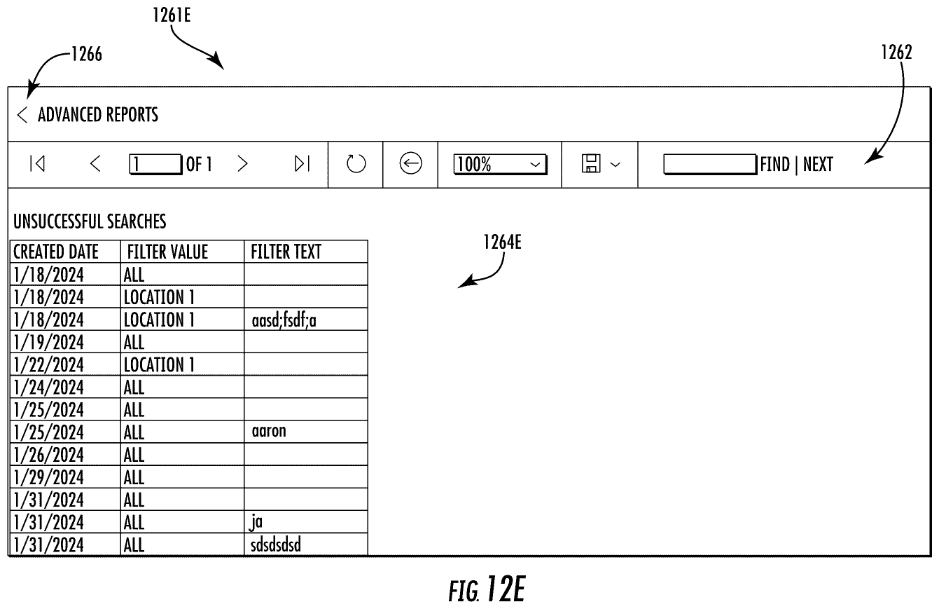

FIG. 9B is an enhanced view illustrating the first menu area 936. The first menu area 936 includes a first menu selection element 936A that may allow the administrator to return to a home screen. The first menu area 936 includes a second menu selection element 936B that may allow the administrator to move to a dashboard screen. The first menu area 936 includes a third menu selection element 936C that may allow the administrator to go to a management screen. The first menu area 936 includes a fourth menu selection element 936D that may allow the administrator to go to a tools screen. The first menu area 936 includes a fifth menu selection element 936E that may allow the administrator to go to a screen for processing and approvals. The first menu area 936 includes a sixth menu selection element 936F that may allow the administrator to go to a reports screen where reports may be obtained. Reports similar to those illustrated in FIGS. 12A-12E may be made available at the reports screen. The first menu area 936 includes a seventh menu selection element 936G that may allow the administrator to go to a Short Shoutout Builder screen(s). In some embodiments, once one of the menu selection elements 936A-936G are selected, that respective menu selection element may be highlighted or otherwise emphasized, and the screen corresponding to that menu selection element may be presented. In FIGS. 9A, 10A, and 11A, the Short Shoutout Builder screen(s) are presented, with the user being able to navigate to different specific screens using the second menu area 938.

FIG. 9C is an enhanced view illustrating the second menu area 938 within the screen 934 of FIG. 9A. The second menu area 938 comprises a first menu selection element 938A, a second menu selection element 938B, a third menu selection element 938C, a cancel button 939A, and a publish button 939B. The first menu selection element 938A is for settings, the second menu selection element 938B is for statements, and the third menu selection element 938C is for styles. In the embodiment illustrated in FIG. 9C, the first menu selection element 938A is selected and emphasized relative to other menu selection elements, but other menu selection elements may be emphasized when they are selected. When one of the menu selection elements are selected, material corresponding to that menu selection element may be presented on the screen 934.

The cancel button 939A may be selected by an administrator to undo any changes that were made. The publish button 939B may be selected once the administrator is satisfied with their settings. Once the publish button 939B is selected, the recognition platform system may cause the material presented to the user to be adjusted based on the proposed changes of the administrator (if any were made).

FIG. 9D is an enhanced view illustrating a logo selection area 940 and a search option customization area 942 within the screen 934 of FIG. 9A. The logo selection area 940 allows for a selected logo 940A to be provided. The logo selection area 940 may present the selected logo 940A as it would appear to any users. The administrator may select the replace button 940B to adjust the selected logo 940A if the administrator wants to replace the selected logo 940A. Once the administrator publishes his or her changes, the selected logo 940A may appear in screens presented to the user (see, e.g., elements 106A and 106B of FIG. 1B, 206A of FIG. 2). The selected logo 940A may also be presented in screens visible to the administrator (see, e.g., element 968 of FIG. 9A).

FIG. 9D also illustrates a search option customization area 942. The search option customization area 942 allows for customization of what users may see when searching for someone. For example, the first selection element 942A may be selected to adjust whether a last name or a last initial of workers are shown in search results, the second selection element 942B may be selected to adjust whether profile images for workers are shown in results, and the third selection element 942C may be selected to adjust whether job titles for workers are shown in results. Based on the selections made by the administrator in the search option customization area 942, the worker icons 324 of FIG. 3D and the worker icons 424 of FIG. 4 may be presented differently. For example, the worker icons 324 of FIG. 3D may be adjusted so that the avatar 324B is shown, is not shown, or so that actual images of the workers are or are not used for the avatar 324B. As another example, the worker icons 324 of FIG. 3D may be adjusted so that the worker identifier 324C includes a last name or last initial of the worker and/or so that a job title for the worker may be presented. While three selection elements 942A-942C are illustrated in FIG. 9D, a different number of selection elements may be utilized in other embodiments, and the selection elements may be used to make selections related to other information (e.g., whether location of worker is shown).

FIG. 9E illustrates an enhanced view of the email option customization area 944 within the screen 934 of FIG. 9A. The email option customization area 944 includes a text field 944A explaining how to use the first input field 944B and the second input field 944C. The text field 944A explains that the material input into the first input field 944B is what the recipient (e.g., the worker) may see in the subject line for the email. The text field 944A also explains that the material input into the second input field 944C is what may be included in the body of every email including a note. In other words, the second input field 944C allows the administrator to add language that may be included in all emails, regardless of the content selected by the user during the note process. The email option customization area 944 may also allow for administrators to manage who may receive a copy of the email (e.g., the worker, the manager or supervisor of the worker and/or others).

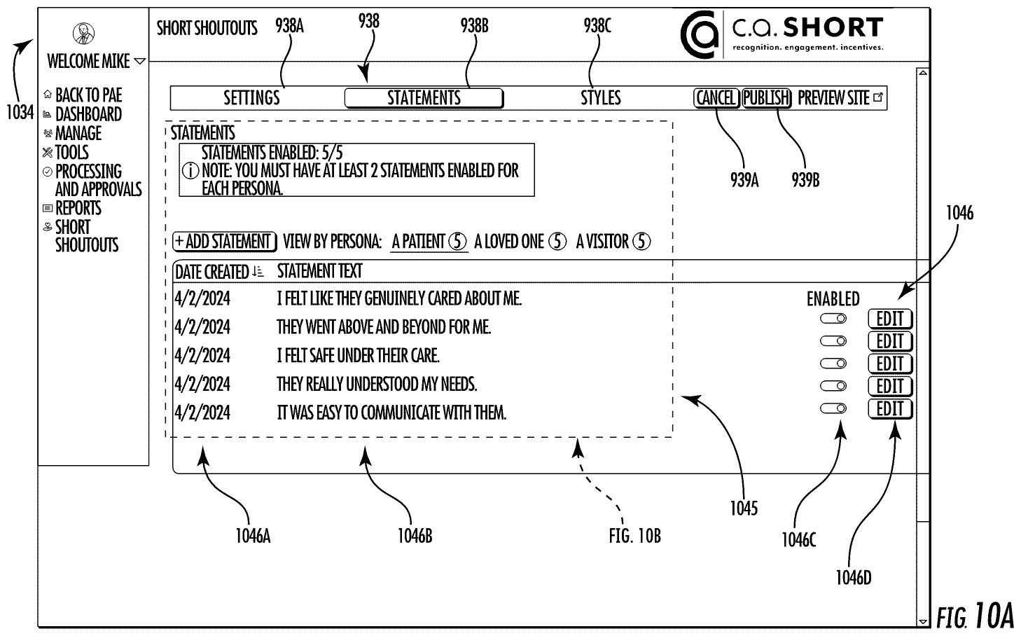

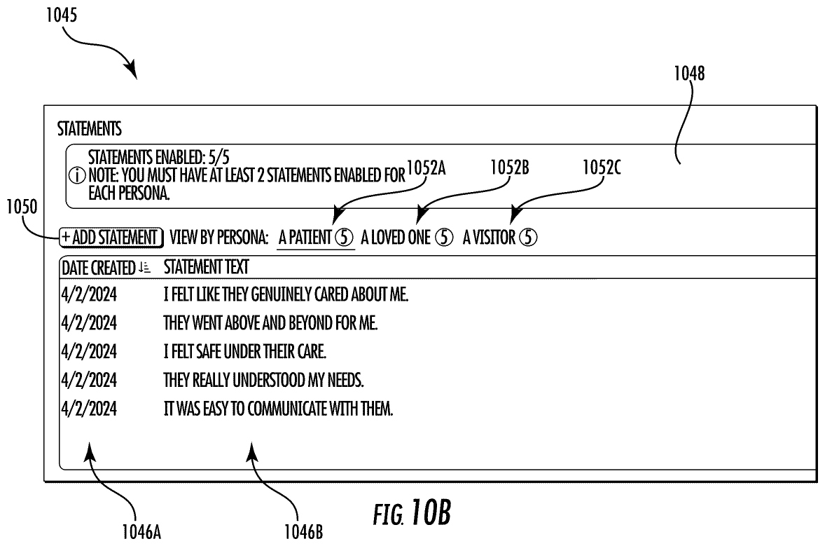

FIG. 10A illustrates an example screen 1034 that may be presented on a display of a user device allowing an administrator to view and/or adjust statements that are presented to users, and FIG. 10B is an enhanced view of a statement area 1045 of the screen 1034 of FIG. 10A. The screen 1034 may be reached by selecting the second menu selection element 938B of the second menu area 938.

A table 1046 is included in the screen 1034, with the table 1046 extending partially into the statement area 1045. The table 1046 includes a first column 1046A, a second column 1046B, a third column 1046C, and a fourth column 1046D. The first column 1046A presents the date that each statement was created, the second column 1046B provides the full statement text for each statement, the third column 1046C indicates whether each statement is currently enabled or not, and the fourth column 1046D includes selectable buttons that are configured to be selected by a user to edit one of the statements. The table 1046 includes five rows because there are five different statements. In other embodiments, the administrator may add up to ten statements in the table 1046 for each persona, and the administrator may be limited to enabling only five statements at any given time. However, a different number of rows and statements may be used, and any number of statements may be enabled at any given time in other embodiments. Additionally, the table 1046 may include a different number of columns in some embodiments.

As illustrated in FIG. 10B, three persona selection buttons 1052A-1052C are illustrated. The first persona selection button 1052A is associated with a selected user type of patient, the second persona selection button 1052B is associated with a selected user type of a loved one, and the third persona selection button 1052C is associated with a selected user type of a visitor.

In FIGS. 10A-10B, the first persona selection button 1052A is selected, so a table is presented with statements corresponding to the first persona (e.g., a patient) associated with the first persona selection button 1052A. Thus, when the user indicates that he or she is a patient (e.g., by selecting first selection element 114A of FIG. 1C), enabled statements appearing in table 1046 may be presented to the user on a subsequent screen (see, e.g., FIG. 5G). However, when the second persona selection button 1052B or the third personal selection button 1052C are selected, the statements appearing in the table 1046 may be associated with that persona (e.g., a loved one or a visitor).

A text field 1048 provides an explanation on the required number of statements for each persona and the limit (if any) on the number of statements for each persona. In the illustrated embodiment, at least two statements must be provided for each persona and no more than five statements are provided for each persona. However, the minimum and maximum number of statements may be adjusted in other embodiments. Statement button 1050 may be selected to add a statement.

In some embodiments, the statements listed in the second column 1046B of the table 1046 may be generated through the use of artificial intelligence (e.g., generative artificial intelligence) using an artificial intelligence unit in some embodiments. By using artificial intelligence, statements may be automatically used that are most appealing to users, that are most appropriate, or that are the best under some other criteria. This may reduce the amount of time that the administrator must take to make the system available to the public. Alternatively, other computer based approaches may be used to automatically generate statements. In some embodiments, artificial intelligence or other computer based approaches may merely generate suggested statements for use in statement selection elements and an administrator may select statements from the suggested statements that the administrator would like to use. For example, the administrator may select the statement button 1050, and one or more suggested statements may be generated through generative artificial intelligence techniques that the administrator may choose to add.

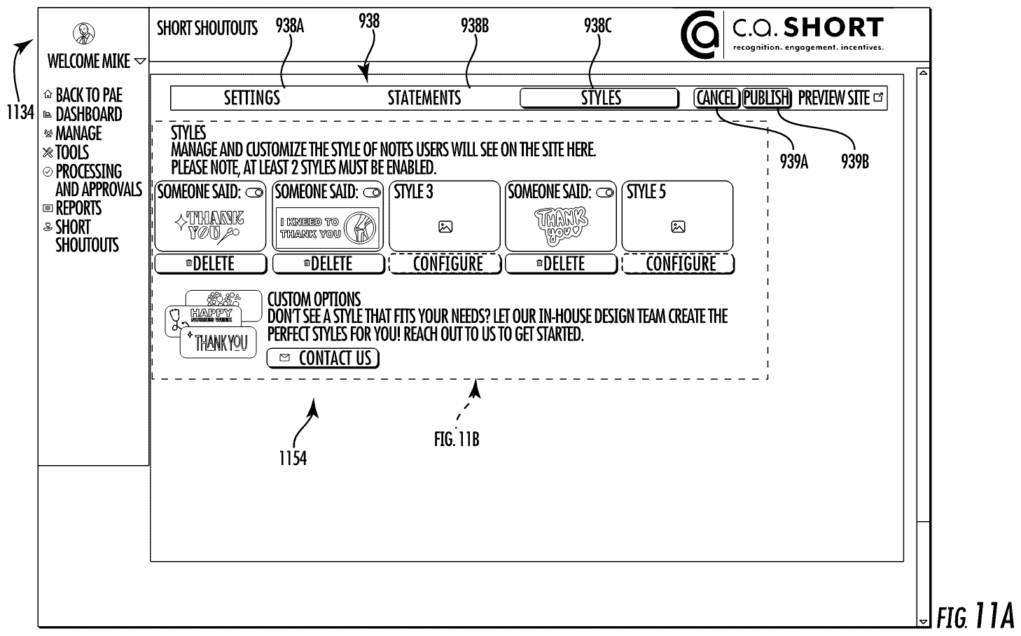

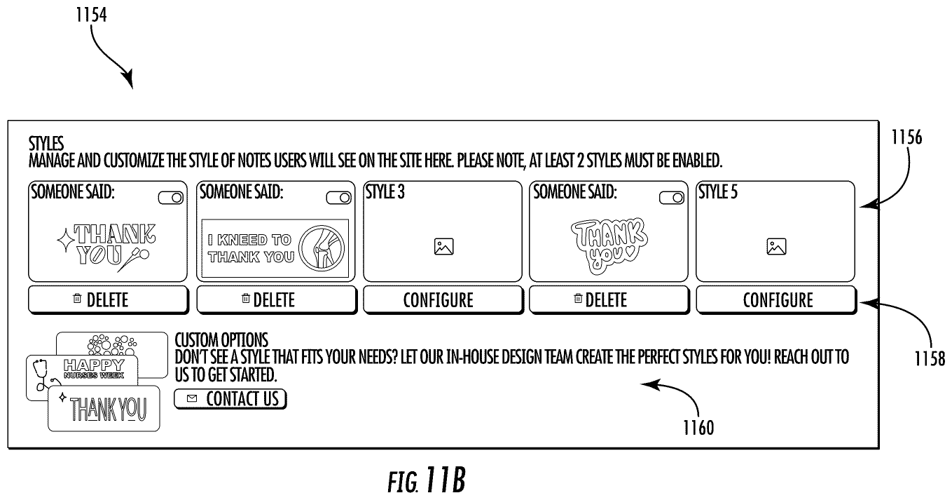

FIG. 11A illustrates an example screen 1134 that may be presented on a display of a user device allowing an administrator to view and/or adjust styles that are available for selection by users, and FIG. 11B is an enhanced view of a style area 1154 of the screen 1134 of FIG. 11A. The screen 1134 may be reached by selecting the third menu selection element 938C of the second menu area 938. As illustrated in FIG. 11B, the style area 1154 may include various style elements 1156 that the user may choose from. In the illustrated embodiment, only three styles elements 1156 are provided for the user to select from, but a different number of styles elements 1156 may be made available in other embodiments. The style area 1154 allows the administrator to select up to five different styles in the illustrated embodiment, but the maximum number of styles made available to the user may be different in other embodiments. Buttons 1158 may be positioned adjacent to each of the style elements 1156. The buttons 1158 may allow the user to configure a style element, to edit an existing style element, or to delete an existing style element. Additional material 1160 at the bottom of the style area 1154 enables the administrator to contact the system operator by selecting a button.

In some embodiments, the styles shown in the style elements 1156 of the style area 1154 may be generated through the use of artificial intelligence (e.g., generative artificial intelligence) using an artificial intelligence unit in some embodiments. By using artificial intelligence, styles may be automatically used that are most appealing to users, that are most appropriate, or that are the best under some other criteria. This may reduce the amount of time that the administrator must take to make the system available to the public. Alternatively, other computer based approaches may be used to automatically generate styles. In some embodiments, artificial intelligence or other computer based approaches may merely generate suggested styles for use in style elements 1156 and an administrator may select styles from the suggested styles that the administrator would like to use. For example, one or more styles may be generated through generative artificial intelligence techniques that the administrator may choose to add.

FIGS. 12A through 12E provide various example reports that may be provided to an administrator. The reports may provide administrators with usage data to make decisions on what statements, styles, and other information they should potentially keep or remove. Additionally, in some embodiments, artificial intelligence units or other computer systems may automatically remove, adjust, or add styles, statements, etc. based on usage information in reports.

The reports may be obtained by selecting the sixth menu selection element 936F of FIG. 9B and by then selecting the desired report from the resulting screen. For each of the report screens presented in FIGS. 12A-12E, a back button 1266 and a menu area 1262 are provided. The back button 1266 allows the administrator to return to a previous screen while the menu area 1262 provides various options for the administrator. The menu area 1262 allows the user to navigate through available reports, to refresh reports, to change the zoom level, to save the reports or download the reports, and to search within the reports. However, the menu area 1262 may be different in other embodiments.

The report screen 1261A of FIG. 12A provides a report on the statement count by employee. The report screen 1261A includes a table 1264A. The table 1264A includes multiple rows, with each row corresponding to a particular worker. The first and second columns provide the worker's first and last name. The third column provides a participant identification number for each worker, the fourth column provides an email address for each worker, and the fifth column provides the organization level for each worker.

The sixth, seventh, and eighth columns of the table 1264A provide information on the number of statements that the employee has received. The sixth column provides an indication of the number of times that the worker received the statement “I feel like they genuinely cared about my relative.” The seventh column provides an indication of the number of times that the worker received the statement “It was easy to communicate with them.” The eighth column provides an indication of the number of times that the worker received the statement “My relative felt safe under their care.” Columns six through eight indicate that John Doe has received note(s) saying that “I feel like they genuinely cared about my relative” and that “My relative felt safe under their care.” Columns six through eight also indicate that Jane Doe received note(s) saying that “It was easy to communicate with them” and that “My relative felt safe under their care.” While only two workers and three different statements are listed in the table 1264A, a different number of workers and statements may be presented in the table 1264A in other embodiments. In some embodiments, artificial intelligence techniques may be used to analyze received data. Artificial intelligence techniques may provide insights into trends or patterns in data to identify facilities (e.g., hospitals), departments, floors, areas, workers, doctors, health professionals, or other workers that are performing at a high level based on feedback from users. In some embodiments, the artificial intelligence techniques may identify patterns that humans and other computer systems may not recognize, thereby allowing high performers to be identified with enhanced accuracy.

The report screen 1261B of FIG. 12B provides a report on the statement count by persona. The report screen 1261B includes a table 1264B. The table 1264B includes multiple rows, with each row corresponding to a particular statement. The first row corresponds to the statement “I felt like they genuinely cared about me,” the second row corresponds to the statement “They went above and beyond for me,” the third row corresponds to the statement “I felt safe under their care,” and the fourth row corresponds to the statement “They really understood my needs.” The first column of the table provides the relevant statements. The second column provides the date that the statements were created, and the third column provides the current status of the statement to indicate whether the statement is disabled or enabled. The fourth column through the sixth columns provide a count of the number of times the statement has been used by different personas. The fourth column indicates the number of times that a person with the user type of “patient” selected a particular statement, the fifth column indicates the number of times that a person with the user type of “loved one” selected a particular statement, the sixth column indicates the number of times that a person with the user type of “visitor” selected a particular statement. For example, the table 1264B indicates that the statement “They really understood my needs” was selected by twenty people having the “patient” user type, by seven people with the “loved one” user type, and by three people having the “visitor” user type.

The report screen 1261C of FIG. 12C provides a report on the number of times each style was used. The report screen 1261C includes a table 1264C. The table 1264C includes multiple rows, with each row corresponding to a particular style (which may include a particular graphic). The first column of the table provides the first line for the notes, the second column provides the graphics included in the notes, the third column provides the date that the style was created, the fourth column indicates whether the style is currently enabled or disabled (i.e., whether or not users may currently use the style), and the fifth column indicates the number of times that the particular style was used. The table 1264C indicates that the count total for the first style of the first row is 25, that the count total for the second style of the second row is 40, and that the count total for the third style of the third row is 4. While the table 1264C does not include separate columns for each persona, the table 1264C may be adjusted to include these separate columns in other embodiments.

The report screen 1261D of FIG. 12D provides a report on the number of notes that each worker received, with the note count being broken down based on user type. The report screen 1261D includes a table 1264D. The table 1264D includes multiple rows, with each row corresponding to a particular worker. For example, John Doe is listed in the first row, Jane Doe is listed in the second row, and James Doe is listed in the third row. The first and second columns of the table 1264D provide the first name and the last name for the worker. The third column provides participant numbers for each worker, the fourth column provides email addresses for each worker, and the fifth column provides the organization level for each worker. The sixth column through the eighth column provides the number of notes that each worker received from different persona types, and the ninth column provides the total number of notes that each worker received. For example, the table 1264D shows that John Doe received one note from a loved one, six notes from patients, two notes from a visitor, and a total of nine notes. The tenth column provides the manager's name and the eleventh column provides the manager's email address.

The report screen 1261E of FIG. 12E provides a report on unsuccessful searches. The unsuccessful searches may be searches that were conducted by the user where the user never selects a worker (or a worker icon associated with a worker). The report screen 1261E includes a table 1264E. The table 1264E includes multiple rows, with each row corresponding to a particular unsuccessful search attempt. The first column of the table 1264E indicates the date when the unsuccessful search was made, the second column of the table 1264E indicates the filter value selected by the user (e.g., in field 322A of FIG. 3C), and the third column of the table 1264E indicates the text that was input by the user (e.g., in input field 322B of FIG. 3C). The administrator may use the report screen 1261E to better organize the worker icons 324 (see FIG. 3D) that are presented to the user. However, in other embodiments, an artificial intelligence unit or another computer system may utilize the information on the report screen 1261E to improve the recognition platform system or to provide suggested improvements.

While various screens are described herein and illustrated in the figures, the screens may be presented differently in other embodiments. For example, elements on the screens may be repositioned, resized, etc. Additionally, additional material may be added to the screens, and some of the material shown on screens may be removed. While the screens are described as being presented in a certain order, the screens may be presented in different orders in other embodiments.

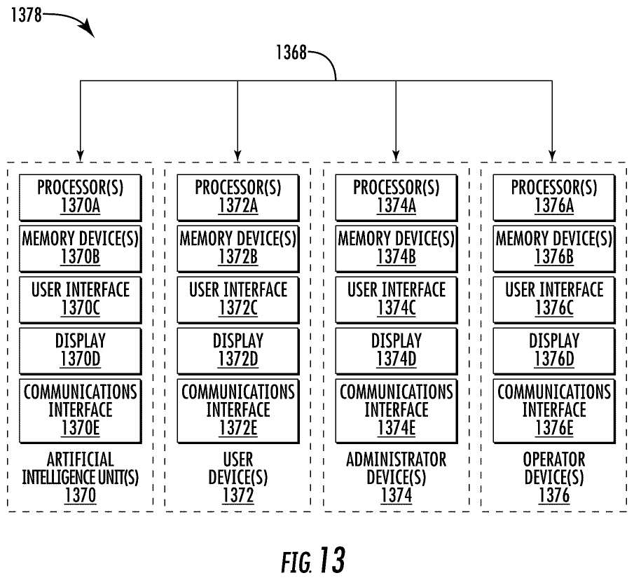

FIG. 13 is a block diagram illustrating various components of an example recognition platform system 1378. The recognition platform system 1378 may be connected to each other using a wired or wireless connection as indicated by the line 1368. The recognition platform system 1378 comprises one or more user devices 1372, one or more administrator devices 1374, one or more operator devices 1376, and one or more artificial intelligence units 1370. In some embodiments, the example platform system 1378 may include one or more servers connected to the line 1368, and the server(s) may include processor(s) and/or memory device(s). Where server(s) are included, these server(s) may be configured to perform some of the functions described herein. However, separate server(s) may be omitted from the recognition platform system 1378 in other embodiments.

The artificial intelligence unit(s) 1370 may comprise one or more processors 1370A, one or more memory devices 1370B, a user interface 1370C, a display 1370D, and a communications interface 1370E. However, in other embodiments, the artificial intelligence unit(s) 1370 may not have a user interface 1370C or a display 1370D. Where this is the case, information from the artificial intelligence unit(s) may be provided to another display (e.g., display 1376D) and commands may be provided to the artificial intelligence unit(s) 1370 from another device (e.g., operator device(s) 1376).

The artificial intelligence unit 1370 may apply artificial intelligence and/or machine learning techniques, and the artificial intelligence unit 1370 may utilize large language models to generate language similar to how humans talk and write. The artificial intelligence unit 1370 may also use the large language models to generate styles and/or graphics. The artificial intelligence unit 1370 may also be used to create other material or to improve existing materials. Use of the artificial intelligence unit 1370 may provide various benefits beyond the benefits provided by basic computing systems. For example, in some embodiments, the artificial intelligence unit 1370 may be used to update models over time as more data is obtained. As such, updated models may be made more accurate because they are based on a larger sample size and because they include the most recent data points, which may be entitled to greater weight than older data points in some situations. The artificial intelligence unit 1370 may also identify patterns that humans and other computer systems may not recognize.

The user device(s) 1372 may be devices utilized by end users to go through the note process. The user device(s) 1372 may include one or more processors 1372A, one or more memory devices 1372B, a user interface 1372C, a display 1372D, and a communications interface 1372E. The administrator device(s) 1374 may be devices utilized by administrators (e.g., workers at a particular company) to set up recognition platform system 1378. The administrator device(s) 1374 may include one or more processors 1374A, one or more memory devices 1374B, a user interface 1374C, a display 1374D, and a communications interface 1374E. The operator device(s) 1376 may be devices utilized by operators. These operators may have even greater access to aspects of the recognition platform system 1378 than the administrators. Administrators may have their visibility limited to relevant data, reports, and information related to their company, while operators may have visibility of information for various companies so that operators may address issues that the administrators may have. The operator device(s) 1376 may include one or more processors 1376A, one or more memory devices 1376B, a user interface 1376C, a display 1376D, and a communications interface 1376E.

The processors 1370A, 1372A, 1374A, 1376A may be any means configured to execute various programmed operations or instructions stored in a memory device (e.g., memory devices 1370B, 1372B, 1374B, 1376B) such as a device or circuitry operating in accordance with software or otherwise embodied in hardware or a combination of hardware and software (e.g. a processor operating under software control or the processor embodied as an application specific integrated circuit (ASIC) or field programmable gate array (FPGA) specifically configured to perform the operations described herein, or a combination thereof) thereby configuring the device or circuitry to perform the corresponding functions of the processors 1370A, 1372A, 1374A, 1376A as described herein. In some embodiments, one or more of the processors 1370A, 1372A, 1374A, 1376A may comprise at least one of a central processing unit, a graphical processing unit, or a tensor processing unit.

The memory devices 1370B, 1372B, 1374B, 1376B may include one or more non-transitory storage or memory devices such as, for example, volatile and/or non-volatile memory that may be either fixed or removable. The memory devices 1370B, 1372B, 1374B, 1376B may be configured to store instructions, computer program code, and/or data in a non-transitory computer readable medium for use, such as by the processors 1370A, 1372A, 1374A, 1376A, for enabling the components of the recognition platform system 1378 to carry out various functions in accordance with example embodiments of the present invention. For example, the memory devices 1370B, 1372B, 1374B, 1376B may be configured to buffer input data for processing by the processors 1370A, 1372A, 1374A, 1376A. Additionally or alternatively, the memory devices 1370B, 1372B, 1374B, 1376B may be configured to store instructions for execution by the processors 1370A, 1372A, 1374A, 1376A. The memory devices 1370B, 1372B, 1374B, 1376B may include computer program code that is configured to, when executed, cause processors 1370A, 1372A, 1374A, 1376A to perform various methods described herein. The memory devices 1370B, 1372B, 1374B, 1376B may serve as non-transitory computer readable mediums having stored thereon software instructions that, when executed by one or more processors, cause methods described herein to be performed.

The user interfaces 1370C, 1372C, 1374C, 1376C may include, for example, a keyboard, keypad, function keys, buttons, a mouse, a scrolling device, input/output ports, a touch screen, or any other mechanism by which a user, administrator, or operator may interface with the recognition platform system 1378.

The displays 1370D, 1372D, 1374D, 1376D (which may each be one or more screens) may be configured to present images and may include or otherwise be in communication with user interfaces 1370C, 1372C, 1374C, 1376C. The displays 1370D, 1372D, 1374D, 1376D may be, for example, a conventional LCD (liquid crystal display), a touch screen display, mobile device display, or any other suitable display known in the art upon which images may be displayed.

The communications interfaces 1370E, 1372E, 1374E, 1376E may be configured to enable communication to other components of the recognition platform system 1378 (e.g. via an external network). Various types of data may be transmitted or received at the communication interfaces 1370E, 1372E, 1374E, 1376E. The communications interfaces 1370E, 1372E, 1374E, 1376E may also include one or more communications modules configured to communicate with one another in any of a number of different manners including, for example, via a network. In this regard, the communications interfaces 1370E, 1372E, 1374E, 1376E may include any of a number of different communication backbones or frameworks including, for example, radio communication Ethernet, global positioning system (GPS), cellular, Wi-Fi, or other suitable networks. In this regard, numerous other peripheral devices may be included in the recognition platform system 1378. In some embodiments, some or all of the communications interfaces 1370E, 1372E, 1374E, 1376E, may be configured to communicate using short-range wireless technologies such as Bluetooth, Wi-Fi, NearLink, near-field communication (NFC), low power wide area networks (LPWAN), ultra-wideband (UWB), wireless local area network (WLAN) in accordance with IEEE 802.11(b), IEEE 802.11(g), and/or IEEE 802.11(n) standards, and/or low-rate wireless personal access networks (LR-WPAN) pursuant to the IEEE 802.15.4 standard. However, devices of the recognition platform system 1378 may be connected by physical wires or connections and may communicate using these wired connections rather than using wireless communications in some embodiments.

Additionally, in some embodiments, artificial intelligence units or other computer systems may automatically remove, adjust, or add styles, statements, etc. based on usage information. This usage information may be limited to the usage information for the administrator's company in some embodiments. However, in other embodiments, the usage information may be pooled from a larger user base for a larger group of companies. Therefore, artificial intelligence units and/or other computer systems may be better equipped to accurately predict which statements and styles may be successful. Where data is pooled from a larger group of companies, any personal identifying information or health information may be obfuscated or removed.

The techniques described above and below may be implemented, for example, in hardware, one or more computer programs tangibly stored on one or more computer-readable media, firmware, or any combination thereof. The techniques described herein may be implemented in one or more computer programs executing on (or executable by) a programmable computer or electronic or computing device having any combination of any number of the following: a processor, a storage medium readable and/or writable by the processor (including, for example, volatile and non-volatile memory and/or storage elements and non-transitory mediums), an input device, an output device, a display, and the like. Program code may be applied to input entered using the input device to perform the functions described herein and to generate output using the output device.

While various components are illustrated in the block diagram of the recognition platform system 1378 of FIG. 13, this block diagram is merely exemplary. In some embodiments, additional components may be added to the recognition platform system 1378 or certain components may be removed from the recognition platform system 1378.

FIG. 14 is a flow chart illustrating an example computer-implemented method 1400 for the creation of recognition notes to a worker. At operation 1402, presentation of a screen is caused, with the screen having selectable elements for user types. This screen may be similar to screen 100 of FIG. 1A or screen 200 of FIG. 2. At operation 1404, a user-type selection may be received once the user has made a selection on the screen that was presented at operation 1402.

At operation 1406, presentation of a screen is caused, with the screen having one or more selectable worker icons. This screen may be similar to screen 300 of FIG. 3A or screen 400 of FIG. 4. At operation 1408, an identification of a worker may be received once the user has made a selection on the screen that was presented at operation 1406.

At operation 1410, presentation of a screen is caused, with the screen having statements available for use in notes. This screen may be similar to screen 500 of FIG. 5A or screen 600 of FIG. 6. At operation 1412, a user selection is received of one or more statements for use in the note once the user has made a selection on the screen that was presented at operation 1410.

At operation 1414, presentation of a screen is caused, with the screen having styles available for notes. This screen may be similar to screen 700 of FIG. 7A or screen 800 of FIG. 8. At operation 1416, a user selection is received for the style of a note once the user has made a selection on the screen that was presented at operation 1414.

At operation 1418, the note may be generated based on the received materials and then sent to the identified worker(s). Notes generated by the recognition platform system may then be sent to the relevant worker via email, text, or as a physical card that may be mailed or otherwise delivered to the relevant worker.

The method 1400 is merely exemplary, and the method 1400 may be modified in various ways. For example, additional operations may be added to method 1400, or certain operations may be omitted from method 1400. Also, the order of operations may be altered in some embodiments. For example, worker(s) may be identified before any user type is received. Additionally, some of the operations may be performed simultaneously.

### CONCLUSION

Many modifications and other embodiments set forth herein will come to mind to one skilled in the art to which these embodiments pertain having the benefit of the teachings presented in the foregoing descriptions and the associated drawings. Therefore, it is to be understood that the embodiments are not to be limited to the specific embodiments disclosed and that modifications and other embodiments are intended to be included within the scope of the invention. Moreover, although the foregoing descriptions and the associated drawings describe example embodiments in the context of certain example combinations of elements and/or functions, it should be appreciated that different combinations of elements and/or functions may be provided by alternative embodiments without departing from the scope of the invention. In this regard, for example, different combinations of elements and/or functions than those explicitly described above are also contemplated within the scope of the invention. Although specific terms are employed herein, they are used in a generic and descriptive sense only and not for purposes of limitation.

That which is claimed:

1. A system for the generation of recognition notes to workers, the system comprising:
a display;
one or more processors; and
one or more memory devices comprising computer program code that is configured, when executed, to cause the one or more processors to:
cause presentation of one or more selectable worker icons on the display;
receive an indication of a selected worker icon;
cause presentation of at least two selectable statements on the display;
receive an indication of a selected statement;
generate a note directed to a worker associated with the selected worker icon,
wherein the note includes the selected statement; and
cause the note to be sent to the worker,
wherein a selectable statement of the at least two selectable statements is generated using an artificial intelligence model.

2. The system of claim 1, further comprising an artificial intelligence unit configured to use a machine learning architecture to generate the artificial intelligence model, wherein the artificial intelligence unit comprises an input layer including a plurality of neurons, one or more hidden layers, with each hidden layer of the one or more hidden layers including a plurality of neurons, and an output layer having a one or more neurons.

3. The system of claim 2, wherein the artificial intelligence unit comprises at least one of a central processing unit, a graphical processing unit, a tensor processing unit, a field-programmable gate array, or an application-specific integrated circuit.

4. The system of claim 1, wherein the computer program code is configured, when executed, to cause the one or more processors to:
cause presentation of at least one selectable user type element; and
receive an indication of user type selection.

5. The system of claim 4, wherein the at least two selectable statements depend on the indication of the user type selection that is received.

6. The system of claim 1, further comprising:
an administrator device configured to adjust the one or more selectable worker icons, the at least two selectable statements, or the at least one selectable style element.

7. The system of claim 1, further comprising an artificial intelligence unit configured to generate a model to identify or create at least one of a statement, a style, or a graphic for use in the note.

8. The system of claim 7, wherein the artificial intelligence unit is configured to train the model using training data and deployed data, and wherein the deployed data is obtained after the model has been trained and deployed.

9. The system of claim 8, wherein the model is a large language model.

10. The system of claim 9, wherein the artificial intelligence unit is configured to:
receive context information; and
identify or create the statement, the style, or the graphic for use in the note based on the context information,
wherein the context information includes at least one of a user type, a location, the selected worker icon, or a role for a worker associated with the selected worker icon.

11. A system for the generation of recognition notes to workers, the system comprising:
a display;
one or more processors; and
one or more memory devices comprising computer program code that is configured, when executed, to cause the one or more processors to:
cause presentation of one or more selectable worker icons on the display;
receive an indication of a selected worker icon;
cause presentation of at least two selectable statements on the display;
receive an indication of a selected statement;
generate a note to a worker associated with the selected worker icon, wherein the note includes the selected statement; and
cause the note to be sent to the worker.

12. The system of claim 11, wherein the computer program code is configured, when executed, to cause the one or more processors to:
receive an indication of two or more selected statements.

13. The system of claim 11, wherein the computer program code is configured, when executed, to cause the one or more processors to:
cause presentation of at least one selectable user type element; and
receive an indication of user type selection.

14. The system of claim 13, wherein the at least two selectable statements depend on the indication of the user type selection that is received.

15. The system of claim 14, wherein the computer program code is configured, when executed, to cause the one or more processors to:
cause presentation of at least one selectable style element; and
receive an indication of a style selection,
wherein the note includes a style associated with the style selection.

16. The system of claim 15, wherein the artificial intelligence unit is configured to train the model using training data and deployed data, and wherein the deployed data is obtained after the model has been trained and deployed.

17. The system of claim 11, further comprising:
an administrator device configured to adjust the one or more selectable worker icons, the at least two selectable statements, or the at least one selectable style element.

18. A method for the generation of recognition notes to workers, the method comprising:
causing presentation of one or more selectable worker icons on the display;
receiving an indication of a selected worker icon;
causing presentation of at least two selectable statements on the display;
receiving an indication of a selected statement;
generating a note to a worker associated with the selected worker icon, wherein the note includes the selected statement; and
causing the note to be sent to the worker.

19. The method of claim 18, further comprising:
causing presentation of at least one selectable user type element; and
receiving an indication of user type selection.

20. The method of claim 18, further comprising:
receiving context information; and
identifying or creating a selectable statement of the at least two selectable statements, a style, or a graphic for use in the note based on the context information,
wherein the context information includes at least one of a user type, a location, the selected worker icon, or a role for a worker associated with the selected worker icon.
